(19) United States
(12) Patent Application Publication
(10) Pub. No.: US 2014/0060145 A1
(43) Pub. Date: Mar. 6, 2014

(54) Analyte Monitoring Methods, Devices and Systems for Recommending Confirmation Tests

(71) Applicant: Abbot Diabetes Care Inc., Alameda, CA (US)

(72) Inventors: Udo Hoss, Castro Valley, CA (US); Marc B. Taub, Mountain View, CA (US); Gary A. Hayter, Oakland, CA (US); Royce Cheng, San Francisco, CA (US); Andrew H. Naegeli, Walnut Creek, CA (US); Christine M. Neuhaus, San Jose, CA (US); Stephen A. Rossi, Hercules, CA (US)

(21) Appl. No.: 14/015,066

(22) Filed: Aug. 30, 2013

Related U.S. Application Data
(60) Provisional application No. 61/695,147, filed on Aug. 30, 2012.

(51) Int. Cl. G01D 18/00; G01N 33/487

(52) U.S. Cl. CPC G01D 18/00; G01N 33/487
USPC 73/1.03

(57) ABSTRACT

In some aspects, methods, devices, and systems for monitoring sensor data and indicating recommendations for confirmation tests on a user interface are provided. Sensor data is received and is monitored to detect predetermined signal characteristics that are associated with a likelihood of inaccuracy of the sensor data. A recommendation for a confirmation test to be performed is indicated on a user interface after the occurrence of a predetermined signal characteristic is detected.

### CROSS-REFERENCE TO RELATED APPLICATIONS

This application claims priority based on U.S. Provisional Application No. 61/695,147, filed Aug. 30, 2012, the disclosure of which is incorporated by reference herein in its entirety.

### BACKGROUND

In many instances it is desirable or necessary to regularly monitor the concentration of particular constituents in a fluid. A number of systems are available that analyze the constituents of bodily fluids such as blood, urine and saliva. Examples of such systems conveniently monitor the level of particular medically significant fluid constituents, such as, for example, cholesterol, ketones, vitamins, proteins, and various metabolites or blood sugars, such as glucose. Diagnosis and management of patients suffering from diabetes mellitus, a disorder of the pancreas where insufficient production of insulin prevents normal regulation of blood sugar levels, requires carefully monitoring of blood glucose levels on a daily basis. A number of systems that allow individuals to easily monitor their blood glucose are currently available. Some of these systems include electrochemical biosensors, including those that include a glucose sensor that is adapted to be positioned in vivo, for example with complete or partial insertion into a subcutaneous site, for example may be transcutaneously positioned, within the body for the continuous in vivo monitoring of glucose levels in bodily fluid (e.g., blood or interstitial fluid (ISF)) of the site.

It is critical that a continuous analyte monitoring system provide accurate and reliable analyte information. Accordingly, it is desirable to have continuous analyte monitoring system devices, systems and methods that provide accurate and reliable analyte information.

### SUMMARY

Continuous analyte monitoring systems, devices and methods self-check or automatically determine if a confirmation of the information obtained from a continuous analyte monitoring system is accurate. The determination is performed in real-time, and is not based on a fixed or static schedule, but rather on the real time information of a system. Confirmation testing based on a fixed or static schedule will likely result in confirmation testing when it is not needed—e.g., when the analyte monitoring signal is operating at high accuracy, such as when there is low or no noise, when the analyte values are not low, when the analyte values are relatively constant or changing at a slow rate, etc.

In some aspects, the subject methods, devices and systems disclosed herein indicate recommendations to perform one or more confirmation tests (e.g., blood test via in vitro test strips, otherwise referred to as “fingersticks”) based on the actual CGM signal, such as during times when the CGM signal is determined to be operating with insufficient accuracy so as to warrant a confirmation test. The recommendation may be informative in some embodiments, and may be required or enforced in other embodiments. Enforcement may include at least temporarily disabling some or all of a continuous analyte monitoring system. The recommendations may be visually and/or audibly and/or vibrationally indicated on a user interface, such as with a display screen, speaker, or vibrational element, respectively. For instance, a recommendation may be helpful to notify the user that a confirmation test should be performed before relying on the results provided by the CGM system—e.g., before therapeutic steps or treatment are performed based on the glucose values, e.g., when a system is integrated or otherwise coupled to a therapy device such as a drug delivery device or the like. One or more of the components of a system may be at least temporarily disabled until a confirmation is test is acknowledged.

In some aspects, the subject methods, devices, and systems may provide recommendations that are based on predetermined signal characteristics of the actual sensor data. The predetermined signal characteristics may be associated with times at which the in vivo sensor data may be inaccurate or likely to be inaccurate. The subject methods, devices, and systems may remove the recommendations at appropriate time, such as when one or more predetermined conditions are met. Examples of predetermined conditions may include one or more of, but are not limited to the completion of a confirmation test, the completion of a confirmation test resulting in a confirmation of accuracy, the elapsing of a predetermined period of time, and an absence of any predetermined signal characteristics detected in the sensor data.

In some aspects of the present disclosure, methods of monitoring in vivo sensor data and indicating recommendations for one or more confirmation tests on a user interface are provided. The method includes receiving sensor data over time; detecting, with processing circuitry, an occurrence of a predetermined signal characteristic in the sensor data; and indicating, on a user interface, a recommendation for a confirmation test after the occurrence of the predetermined signal characteristic is detected. The sensor data is derived from an in vivo positioned analyte sensor, and the predetermined signal characteristic is associated with a likelihood of inaccuracy of the sensor data.

In some aspects of the present disclosure, in vivo analyte monitoring systems for monitoring sensor data and indicating recommendations for one or more confirmation tests on a user interface are provided. The analyte monitoring systems include a user interface programmed to indicate one or more of recommendations for one or more confirmation tests, including but not limited to, processing circuitry operably coupled to the user interface, and memory operably coupled to the processing circuitry. The memory includes instructions stored therein, which when executed by the processing circuitry, cause the processing circuitry to receive sensor data over time, detect an occurrence of a predetermined signal characteristic in the sensor data, and indicate, on a user interface, a recommendation for a confirmation test after the occurrence of the predetermined signal characteristic is detected. The sensor data is derived from an in vivo positioned analyte sensor, and the predetermined signal characteristic is associated with a likelihood of inaccuracy of the sensor data.

### INCORPORATION BY REFERENCE

Additional embodiments of analyte monitoring systems suitable for practicing methods of the present disclosure are described in U.S. Pat. No. 6,175,752, U.S. Pat. No. 6,134,461, U.S. Pat. No. 6,579,690, U.S. Pat. No. 6,605,200, U.S. Pat. No. 6,605,201, U.S. Pat. No. 6,654,625, U.S. Pat. No. 6,746,582, U.S. Pat. No. 6,932,894, U.S. Pat. No. 7,090,756, U.S. Pat. No. 5,356,786; U.S. Pat. No. 6,560,471; U.S. Pat. No. 5,262,035; U.S. Pat. No. 6,881,551; U.S. Pat. No. 6,121,009; U.S. Pat. No. 7,167,818; U.S. Pat. No. 6,270,455; U.S. Pat. No. 6,161,095; U.S. Pat. No. 5,918,603; U.S. Pat. No. 6,144,837; U.S. Pat. No. 5,601,435; U.S. Pat. No. 5,822,715; U.S. Pat. No. 5,899,855; U.S. Pat. No. 6,071,391; U.S. Pat. No. 6,377,894; U.S. Pat. No. 6,600,997; U.S. Pat. No. 6,514,460; U.S. Pat. No. 5,628,890; U.S. Pat. No. 5,820,551; U.S. Pat. No. 6,736,957; U.S. Pat. No. 4,545,382; U.S. Pat. No. 4,711,245; U.S. Pat. No. 5,509,410; U.S. Pat. No. 6,540,891; U.S. Pat. No. 6,730,200; U.S. Pat. No. 6,764,581; U.S. Pat. No. 6,503,381; U.S. Pat. No. 6,676,816; U.S. Pat. No. 6,893,545; U.S. Pat. No. 6,514,718; U.S. Pat. No. 5,262,305; U.S. Pat. No. 5,593,852; U.S. Pat. No. 6,746,582; U.S. Pat. No. 6,284,478; U.S. Pat. No. 7,299,082; U.S. Pat. No. 7,811,231; U.S. Pat. No. 7,822,557; U.S. Pat. No. 8,106,780; U.S. Patent Application Publication No. 2010/0198034; U.S. Patent Application Publication No. 2010/0324392; U.S. Patent Application Publication No. 2010/0326842 U.S. Patent Application Publication No. 2007/0095661; U.S. Patent Application Publication No. 2008/0179187; U.S. Patent Application Publication No. 2008/0177164; U.S. Patent Application Publication No. 2011/0120865; U.S. Patent Application Publication No. 2011/0124994; U.S. Patent Application Publication No. 2011/0124993; U.S. Patent Application Publication No. 2010/0213057; U.S. Patent Application Publication No. 2011/0213225; U.S. Patent Application Publication No. 2011/0126188; U.S. Patent Application Publication No. 2011/0256024; U.S. Patent Application Publication No. 2011/0257495; U.S. Patent Application Publication No. 2012/0157801; U.S. Patent Application Publication No. 2012/0157801; U.S. Patent Application Publication No. 2010/0213057; U.S. patent application Ser. No. 13/407,617; U.S. patent application Ser. No. 13/526,136; U.S. patent application Ser. No. 12/698,124; and U.S. patent application Ser. No. 12/807,278, the disclosures of each of which are incorporated herein by reference in their entirety.

### BRIEF DESCRIPTION OF THE DRAWINGS

A detailed description of various embodiments of the present disclosure is provided herein with reference to the accompanying drawings, which are briefly described below. The drawings are illustrative and are not necessarily drawn to scale. The drawings illustrate various embodiments of the present disclosure and may illustrate one or more embodiment(s) or example(s) of the present disclosure in whole or in part. A reference numeral, letter, and/or symbol that is used in one drawing to refer to a particular element may be used in another drawing to refer to a like element.

FIG. 1 illustrates a flowchart for a method of monitoring sensor data and indicating, with a user interface, recommendations to perform confirmation tests, according to one embodiment.

FIG. 2 illustrates an exemplary analyte meter including a graphical element on the display of the user interface to indicate a recommendation for a confirmation test, such as a fingerstick test using a blood sample, according to one embodiment.

FIG. 3 illustrates an example flowchart for a method of monitoring sensor data and indicating, with a user interface, recommendations to perform confirmation tests, according to one embodiment.

FIG. 4 illustrates a flowchart for a method of monitoring sensor data and indicating, with a user interface, recommendations to perform confirmation tests, according to one embodiment.

FIG. 5A illustrates a plot of an example sensor data collected from an analyte sensor over time, according to one embodiment.

FIG. 5B illustrates a plot of sensor data and corresponding slopes at various points in the sensor data, according to one embodiment.

FIG. 6 shows an analyte (e.g., glucose) monitoring system, according to one embodiment.

FIG. 7 is a block diagram of the data processing unit 602 shown in FIG. 6 in accordance with one embodiment.

FIG. 8 is a block diagram of an embodiment of a receiver/monitor unit such as the primary receiver unit 604 of the analyte monitoring system shown in FIG. 6.

FIG. 9 illustrates an example analyte (e.g., glucose) monitoring system, according to one embodiment.

### DETAILED DESCRIPTION

Before the embodiments of the present disclosure are described, it is to be understood that the present disclosure is not limited to particular embodiments described, as such may, of course, vary. It is also to be understood that the terminology used herein is for the purpose of describing particular embodiments only, and is not intended to be limiting, since the scope of the embodiments of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the present disclosure. The upper and lower limits of these smaller ranges may independently be included or excluded in the range, and each range where either, neither or both limits are included in the smaller ranges is also encompassed within the present disclosure, subject to any specifically excluded limit in the stated range. Where the stated range includes one or both of the limits, ranges excluding either or both of those included limits are also included in the present disclosure.

In the description of the present disclosure herein, it will be understood that a word appearing in the singular encompasses its plural counterpart, and a word appearing in the plural encompasses its singular counterpart, unless implicitly or explicitly understood or stated otherwise. Merely by way of example, reference to “an” or “the” “analyte” encompasses a single analyte, as well as a combination and/or mixture of two or more different analytes, reference to “a” or “the” “concentration value” encompasses a single concentration value, as well as two or more concentration values, and the like, unless implicitly or explicitly understood or stated otherwise. Further, it will be understood that for any given component described herein, any of the possible candidates or alternatives listed for that component, may generally be used individually or in combination with one another, unless implicitly or explicitly understood or stated otherwise. Additionally, it will be understood that any list of such candidates or alternatives, is merely illustrative, not limiting, unless implicitly or explicitly understood or stated otherwise.

Various terms are described below to facilitate an understanding of the present disclosure. It will be understood that a corresponding description of these various terms applies to corresponding linguistic or grammatical variations or forms of these various terms. It will also be understood that the present disclosure is not limited to the terminology used herein, or the descriptions thereof, for the description of particular embodiments. Merely by way of example, the present disclosure is not limited to particular analytes, bodily or tissue fluids, blood or capillary blood, or sensor constructs or usages, unless implicitly or explicitly understood or stated otherwise, as such may vary. The publications discussed herein are provided solely for their disclosure prior to the filing date of the application. Nothing herein is to be construed as an admission that the embodiments of the present disclosure are not entitled to antedate such publication by virtue of prior invention. Further, the dates of publication provided may be different from the actual publication dates which may need to be independently confirmed.

In some aspects, the subject matter disclosed herein relates to continuous analyte monitoring systems, such as continuous glucose monitoring (CGM) systems. The system provides recommendations to perform confirmation testing to the user by indicating the recommendation on a user interface of the system. For example, the recommendation may be visually and/or audibly and/or vibrationally indicated on the user interface, such as with a display screen, speaker, or vibrational element, respectively.

The confirmation test may be a contemporaneously-obtained reference test value to confirm the CGM value. The reference may be a laboratory test or a test designed for testing by a user outside of a laboratory such as an in vitro analyte testing system such as in vitro analyte test strips and associated test strip meters, e.g., blood-derived analyte level confirmations (or other biological fluid). In one embodiment, the confirmation test is a blood-derived confirmation test which is compared against sensor data that is derived from an in vivo positioned sensor that is contacting interstitial fluid (or other) of a test subject. For example, a blood test measurements may be performed by drawing blood from a finger of the patient and applying it to an ex-vivo analyte test strip (e.g., also referred to herein as a “fingerstick”). The analyte test strip is used by its analyte meter device to analyze the blood sample and provide an analyte measurement based on the blood sample. The continuous analyte monitoring may then use one or more blood-derived confirmation tests to ensure accuracy and reliability of CGMS systems. For instance, in one embodiment, one or more blood-derived confirmation tests may be used to confirm the data of the CGM sensor data that is derived from a glucose sensor positioned in vivo into interstitial fluid (ISF) of the body of a patient.

The subject methods, devices, and systems provide recommendations that are based on the actual in vivo sensor data and may be associated with times at which the in vivo sensor data is determined to be inaccurate or likely to be inaccurate. In this way, the CGM system may, in real time, receive sensor data and compare it against predetermined signal characteristics that may be indicative of inaccurate in vivo sensor data, or indicative of potentially inaccurate sensor data. In this way, the recommendation for in vitro confirmation tests may be generated in a dynamic manner rather than a static or fixed manner or schedule. Furthermore, the recommendations may be generated during times where such confirmation testing is necessary.

The recommendations may be indicated on the user interface and removed at the appropriate time, such as when a predetermined condition is met. Example predetermined conditions may include, but are not limited to, one or more of the completion of a confirmation test, the completion of a confirmation test resulting in a confirmation of accuracy, the elapsing of a predetermined period of time, and an absence of any predetermined signal characteristics detected in the sensor data. The predetermined conditions may include a predetermined period of time—e.g., from the detection of the predetermined signal characteristic, from the time the recommendation is indicated with the user interface, from the time a confirmation test is completed, etc. The predetermined period of time may be, for example, 1 hour after the detection of the predetermined signal characteristic, or 2 hours after the detection, or 3 hours after the detection, or other predetermined period of time after detection). In some instances, different predetermined periods of time may be implemented for different predetermined signal characteristics that are detected. Similarly, different predetermined conditions may be implemented for different predetermined signal characteristics that are detected.

In certain embodiments, the recommendation may be informative. In other words, if it is solely informative, it is designed to notify a user, but not to enforce the recommendation (disrupt, change, or interfere with, the analyte monitoring of the system). For instance, a recommendation may be helpful to notify the user that a confirmation test should be performed before relying on the results provided by the CGM system—e.g., before therapeutic steps or treatment are performed based on the glucose values, e.g., when a system is integrated or otherwise coupled to a therapy device such as a drug delivery device or the like.

In certain embodiments, the system may solely or also enforce the recommendation, e.g., by disabling some or all of a continuous analyte monitoring system until certain predetermined conditions are met. For instance, one or more features of the monitoring system may be disabled until the system receives user input verifying that the user has acknowledged the recommendation, at which point the one or more features may be re-enabled either automatically or manually by a user. In some instances, the user input may consist of the user actuating a switch, for example pressing a button, or other input element (e.g., physically on the device, or displayed on a touchscreen) to acknowledge the recommendation. In some instances, the user input may consist of the user performing the recommended confirmation test before the one or more features are activated. For example, the user may perform an in vitro analyte test measurement with an analyte test strip to compare to data of an analyte monitoring system, where the in vitro analyte meter is an integrated component of the in vivo analyte monitoring system. Alternatively, the user may perform an in vitro analyte test measurement with separate in vitro analyte meter and then input the results of confirmation test in the in vivo analyte monitoring system. In one embodiment, enforcement includes disabling a feature associated with providing the user with therapeutic recommendations (e.g., drug administration recommendations such as insulin calculations, etc., based on the sensor data). Enforcement may also include the initiation or deactivation of other measures or features in response to a recommendation.

In certain embodiments, the analyte is glucose and the analyte monitoring device is a glucose monitoring device, such as a glucose meter. While specific references to glucose monitoring systems (e.g., CGM systems) are provided herein, the subject matter and concepts described are not limited thereto, but may also be applicable to analyte monitoring systems in general. Furthermore, while specific references to glucose may be provided herein, the subject matter and concepts of the present disclosure is not limited to glucose, but may also be applicable to other analytes, such as ketone bodies for instance. Furthermore, the subject matter of the present disclosure are not limited to the monitoring of a single analyte, but may also be applicable to the monitoring of more than one analyte—e.g., glucose and ketone bodies.

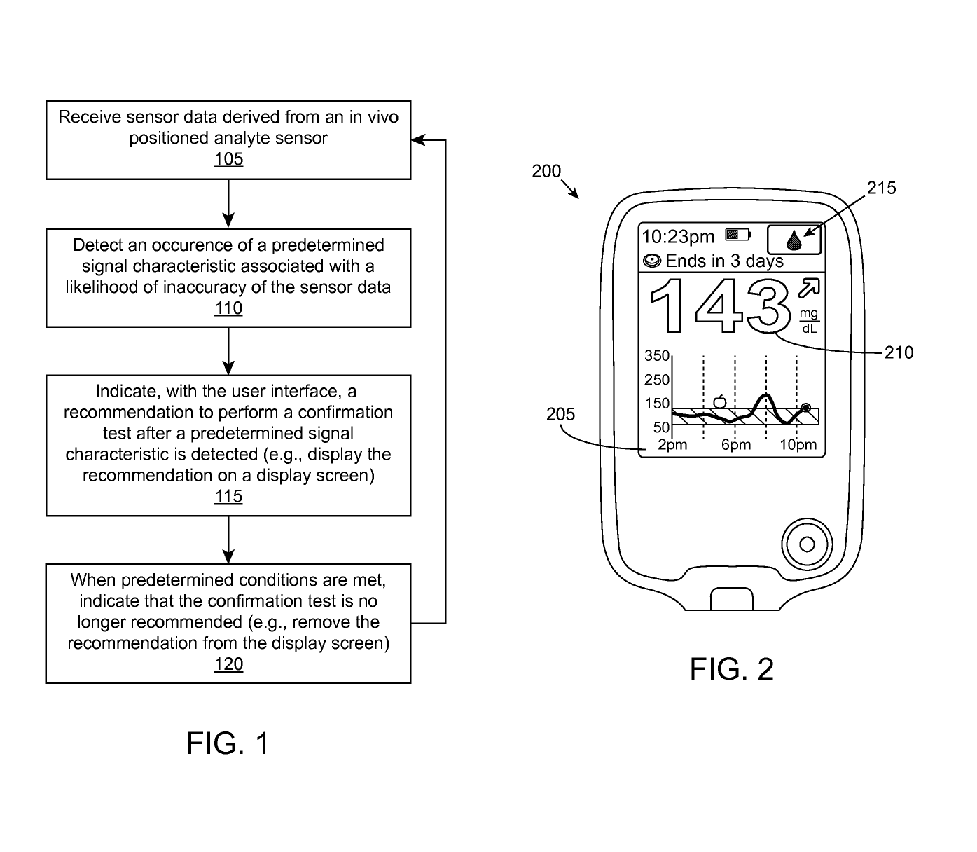

FIG. 1 illustrates a flowchart for a method of monitoring sensor data and indicating, with a user interface, recommendations to perform confirmation tests, according to one embodiment. At block 105, sensor data that is derived from an in vivo positioned analyte sensor is received. For example, the continuous analyte monitoring system may include an analyte sensor having at least a portion of the sensor positioned in vivo beneath the skin surface of a test subject (e.g., human being or other animal) to contact bodily fluid, such as interstitial fluid (ISF), to monitor one or more analytes in the body fluid over a period of time. This is also referred to as continuous analyte monitoring in that the sensor remains positioned in the user for a continuous period of time.

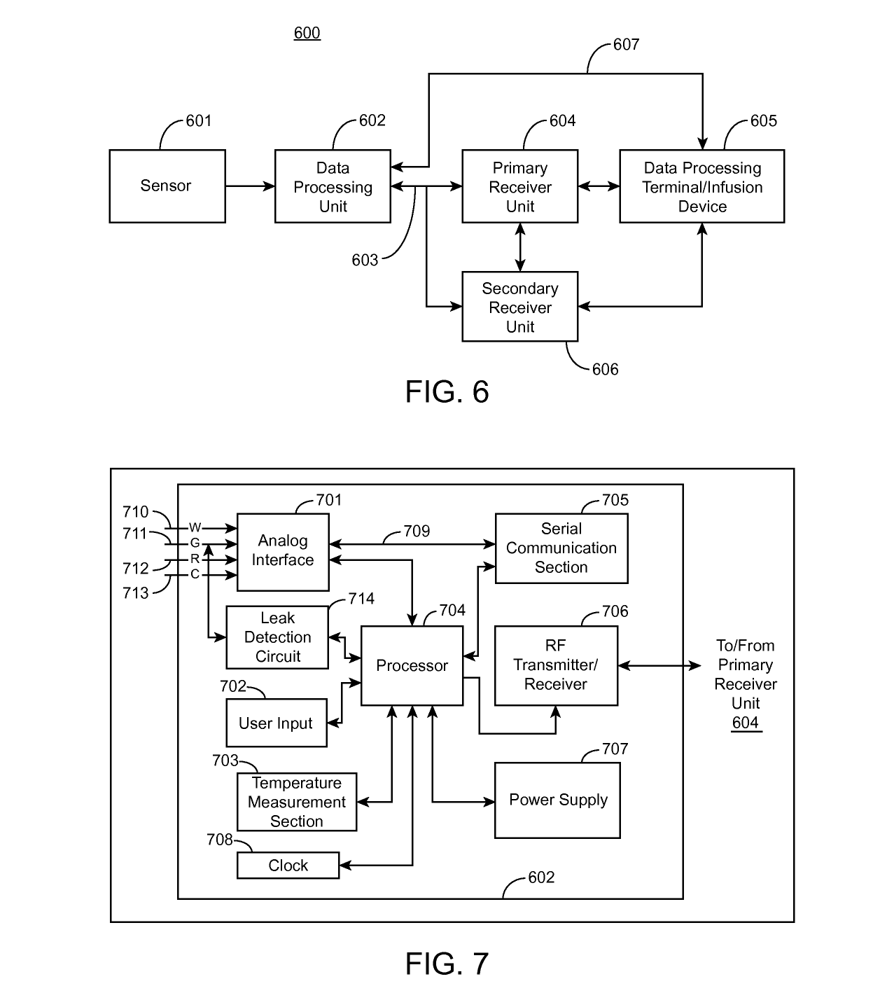

A sensor electronics unit may be coupled to the in vivo positioned sensor and coupled to the body of a patient. The sensor electronics unit receives sensor data from the in vivo analyte sensor and communicates the sensor data (e.g., in raw or processed form) wired or wirelessly to a receiver unit (e.g., an analyte monitoring device including a wireless receiver). For example, the sensor electronics unit may include processing circuitry, memory for storing sensor data collected over time, and a transmitter to transmit the data to the receiver unit. The sensor may include additional electronic components, and further details of example sensors are provided in FIGS. 6-9. The receiver unit (e.g., analyte monitoring device including a wireless receiver) receives the sensor data from the sensor electronics unit and performs the methods described herein, such as with processing circuitry included within the analyte monitoring device. The term “processing circuitry” is used broadly herein to mean any type of circuitry, component, integrated circuit, microchip, etc., that perform arithmetic and logic operations, such as those in response to and processing basic computer instructions. Processing circuitry may refer to, for example, one or more processors, microprocessors, microcontrollers, programmable circuitry programmed or configured by software and/or firmware, special-purpose “hardwired” circuitry such as application-specific integrated circuits (ASICS), programmable logic devices (PLDs), field-programmable gate arrays (FPGAs), etc. In another embodiment, the methods described herein may be performed by processing circuitry of the sensor electronics unit. In yet another embodiment, the methods described herein may be performed in combination between processing circuitry on the sensor electronics unit and processing circuitry coupled to the receiver which communicates with the sensor electronics unit. Other components and devices may also be implemented in the system, such as medication delivery devices (e.g., insulin delivery devices), other input/output devices, etc.

The sensor electronics unit may continuously or periodically transmit sensor data to the receiver unit,—e.g., as may occur with CGM systems. The sensor data may be communicated periodically, such as at a certain frequency as data is obtained or after a certain time period of sensor data is stored in memory. For example, sensor electronics coupled to an in vivo positioned sensor may collect the sensor data for a predetermined period of time and transmit the collected data periodically (e.g., every minute, five minutes, or other predetermined period) to an analyte monitoring device that is positioned in range from the sensor electronics.

The sensor data may include a number of samples taken over time and may be digital data that is representative of a continuous signal. For instance, numerous samples of a test subject's glucose level may be taken over time at a predetermined sampling rate and be representative of the continuous change in glucose level of the test subject over that time period.

In other embodiments, the sensor electronics coupled to the in vivo positioned sensor may communicate with the analyte monitoring device in a non-periodic manner and not set to any specific schedule or frequency. For example, the sensor data may be communicated from the sensor electronics to the analyte monitoring device using RFID technology, and communicated whenever the sensor electronics are brought into communication range of the analyte monitoring device. For example, the in vivo positioned sensor may collect sensor data in memory until the analyte monitoring device (e.g., receiver unit) is brought into communication range of the sensor electronics unit—e.g., by the patient or user. When the in vivo positioned sensor is detected by the analyte monitoring device, the device establishes communication with the sensor electronics and uploads the sensor data that has been collected since the last transfer of sensor data, for instance. In this way, the patient does not have to carry the analyte monitoring device at all times, and instead, can upload sensor data when desired by bringing the analyte monitoring device into range of the analyte sensor. In yet other embodiments, a combination of periodic and non-periodic transfers of sensor data may be implemented in certain embodiments. For example, transfers of sensor data may be initiated when brought into communication range o, and then continued on a periodic basis if continued to remain in communication range.

At block 110 of FIG. 1, an occurrence of a predetermined signal characteristic is detected. The predetermined signal characteristic is associated with a likelihood of inaccuracy in the sensor data. The phrase “associated with a likelihood of inaccuracy in the sensor data” is used herein generally to mean that the predetermined signal characteristic may be indicative of inaccurate sensor data, or indicative of potentially inaccurate sensor data or sensor data that is otherwise likely to be inaccurate. A predetermined signal characteristic may include, but is not limited to, any variety of attributes or characteristics of the sensor data, including various times, events, conditions, etc., that may be related to the sensor data.

For instance, a predetermined signal characteristic may include one or more ranges of analyte levels (e.g., glucose levels)—e.g., a predetermined “low glucose” range (e.g., 90 mg/dL or less, including 70 mg/dL or less, such as 50 mg/dL or less, or other predetermined low glucose range.) which may be more susceptible to signal noise and less accurate (e.g., have higher signal-to-noise ratios). In certain embodiments, a predetermined “high glucose” range (e.g., 180 mg/dL or more, including 200 mg/dL or more, such as 240 mg/dL or more, or 300 mg/dL or more, or other predetermined high glucose range) may be implemented, such as with systems that have been designed to be more accurate at lower glucose levels by giving up some accuracy in the higher glucose levels.

The predetermined signal characteristic may include predetermined ranges of analyte rate-of-change that are associated with a likelihood of inaccuracy. For example, the predetermined signal characteristic may refer to a predetermined signal event, such as a rapidly changing analyte values (e.g., glucose values)—e.g., due to lag in the response time of sensor data derived from interstitial fluid (ISF). For example, a predetermined condition may include sensor data having glucose rates of change larger than a threshold rate-of-change, such as 1 mg/dL per minute or greater, 2 mg/dL per minute or greater, or 3 mg/dL per minute or greater, or other range of glucose rate-of-change. In one embodiment, the predetermined condition includes sensor data having glucose rates of change of 5 mg/dL per minute or greater.

The predetermined signal characteristic may include sensor data that is received within a predetermined time period after sensor insertion—e.g., a predetermined time period related to early sensor attenuation (ESA) when the sensor data may be inaccurate. For example, a predetermined signal characteristic may include sensor data received within the first 5 hours, the first 10 hours, the first 24 hours, or other predetermined time period since sensor insertion. In some instances, the predetermined time period related to early sensor attenuation (ESA) may not begin exactly at the time of sensor insertion or activation, but rather at some related time thereafter. For example, the beginning of the predetermined period of time may begin at some time after insertion or activation (e.g., as 1 hour after sensor insertion, 2 hours after sensor insertion, 4 hours after sensor insertion or other length of time thereafter) based on the empirically observed time course of ESA associated with typical wound response times. Thus, for instance, no confirmation may be needed for the first four hours (0 to 4^{th} hour) after sensor insertion or activation, but then needed from the 4^{th} hour (e.g., associated with the wound response time) until the 24^{th} hour (e.g., the end of the ESA period).

The predetermined signal characteristic may include sensor data that results in a low calculated confidence estimate. For example, an algorithm may be implemented to calculate confidence values for corresponding sensor data (e.g., analyte values, such as glucose values) that relate to a probability, likelihood, or degree of certainty of being accurate.

Furthermore, a predetermined signal characteristic may include sensor data having one or more signal irregularities. The signal irregularity may be indicative of an atypical reading, error, noisy signal, etc. The signal irregularities may appear as deviations from the continuous flow or pattern of the sensor data when the sensor data is plotted. Other signal irregularities may include sensor data having high sensor to noise ratios.

In certain embodiments, a signal irregularity may include signal outliers, signal dropouts, artifacts, or other aberrations, that form a spike, discontinuity, etc., in the sensor data. For example, spikes may be identified as relatively significant changes in analyte values in a short or otherwise brief time, such as that created by signal outliers or dropouts. A spike may include a large positive slope followed by large negative slope, or vice versa, in a short period of time. The thresholds for the size and duration of the slopes may vary in different applications and embodiments. For example, in certain embodiments, example parameters for a spike with regard to CGM systems may include relatively large positive and negative slopes, such as 5 mg/dL per minute or greater (e.g., 6 mg/dL per minute or greater, 7 mg/dL per minute or greater, etc.) within a relatively short period of time, such as fifteen minutes or less (e.g, 5 minutes or less, or one minute or less), or within a relatively short sampling duration, such as 10 samples or less (e.g., 5 samples or less, or 2 samples or less, or 1 sample). The parameters of the spike, and signal irregularities in general, may vary in different embodiments and may be pre-programmed so that the system may identify the spike, or signal irregularity, based on the defined parameters. In certain embodiments, the parameters of the spike, or other signal irregularity, may be defined based on the patient's or user's type of diabetes and/or the treatment modality. For example, someone with Type 1 diabetes may be expected to have higher rates of changes in general than someone with Type 2 diabetes on oral agents, and thus the parameters for a spike for a patient with Type 1 diabetes may be defined at a higher rate than a patient with Type 2 diabetes in order to accommodate the higher rate of change expected of the Type 1 patient. In certain embodiments, the parameters of the spike, or other signal irregularity, may be auto-configured based upon the observed distribution of rates of change for that person.

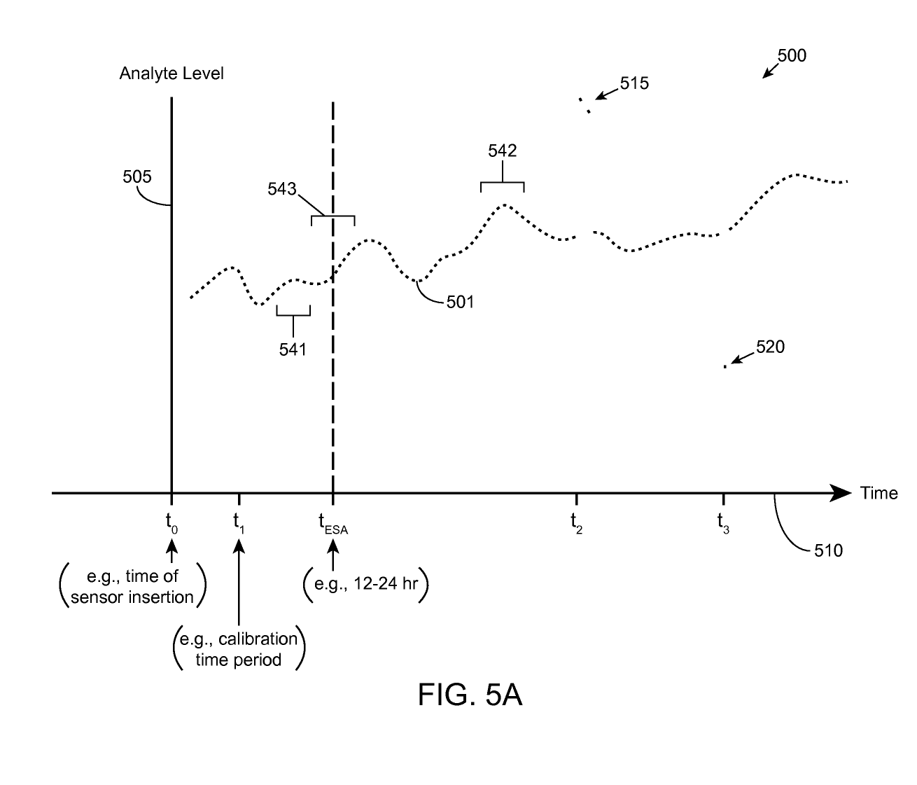

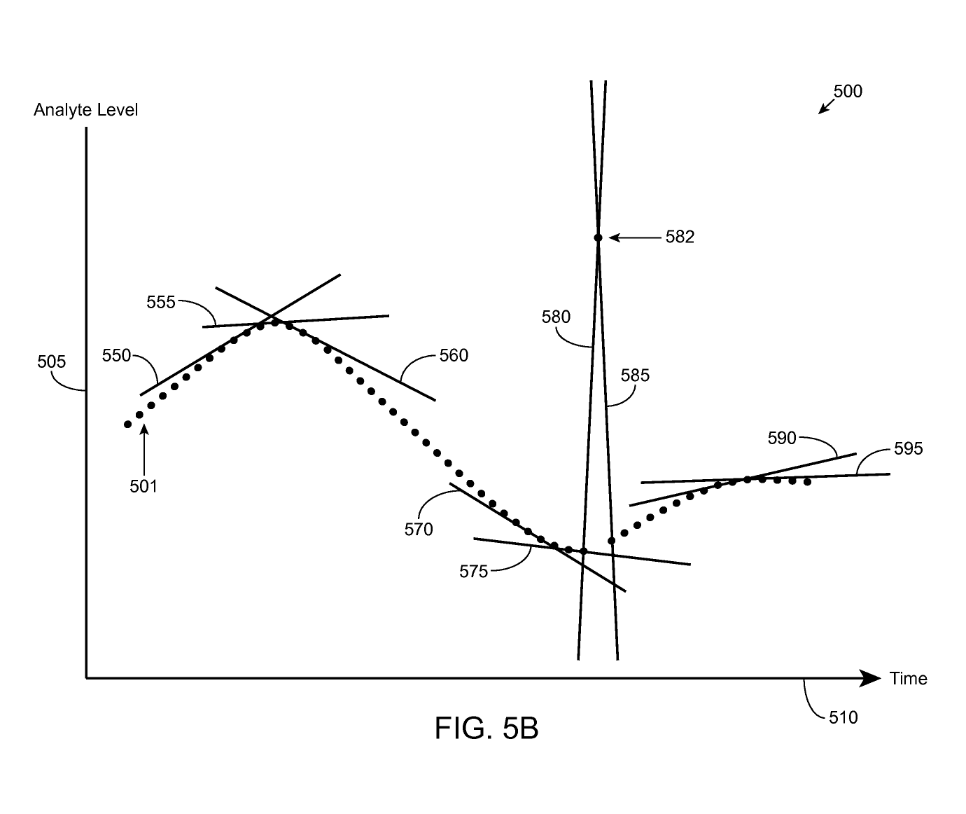

A variety of signal characteristic detection techniques or algorithms may be implemented to identify the predetermined signal characteristic. For example, in one embodiment, a signal irregularity, such as an outlier or dropout, may be identified by analyzing the slope of the sensor data (e.g., glucose values). For example, the detection algorithms may calculate various slopes of the sensor data to determine if any changes in slope are indicative of a spike or discontinuity. For instance, a spike may be representative of a large positive slope followed by an immediate large negative slope, or vice versa, and thus the slope detection algorithm may calculate slopes of the analyte signal data looking for such changes in slope. Furthermore, spikes from outliers and dropouts may also be determined by looking for large jumps in signal value, either positive or negative, within a short period of time. The tolerances and thresholds for the jumps may vary in different applications and embodiments. Additional information regarding slope detection is discussed in FIG. 5B.

The preceding signal irregularities are exemplary, and other irregularities may be implemented. The signal irregularities may include any predetermined or predefined signal anomaly. The predetermined signal characteristics may also vary, for example, for different conditions in different markets—e.g., based upon local regulatory approvals. Furthermore, the preceding predetermined signal characteristics are exemplary, and other predetermined signal characteristics associated with a likelihood of inaccuracy in the sensor data may be implemented.

At block 115, a recommendation to perform a confirmation test is indicated with the user interface after the predetermined signal characteristic is detected. For example, an analyte monitoring system may include a user interface, such as a display (e.g., liquid crystal display (LCD) screen) to indicate the recommendation to perform a confirmation test to the user. For example, the receiver may include the user interface or have the user interface coupled to the receiver. In another embodiment, the user interface may be coupled to the sensor electronics unit and the methods described herein performed by processing circuitry of the sensor electronics unit. In yet another embodiment, the method of FIG. 1 may be performed in combination between processing circuitry on the sensor electronics unit and processing circuitry coupled to the receiver which communicates with the sensor electronics unit.

The recommendation may be displayed in any variety of one or more forms in various embodiments, such as, but not limited to, textual, graphical (e.g., symbols, icons, pictures, images, etc.), a sequence of graphical elements which are displayed in a manner to represent motion (e.g., representing a sequence of steps in motion, such as performing the confirmation test, etc.), video (e.g., short video clips, etc.), etc. For instance, in one embodiment, the recommendation is displayed as an icon, symbol, or other graphical element representing the recommendation to perform the confirmation test. For example, a graphical element representing a confirmation test may be in the form of an icon or image of a blood drop, fingerstick, etc., to indicate a fingerstick confirmation test using a blood sample is recommended. FIG. 2 illustrates an exemplary analyte meter including a graphical element on the display of the user interface to indicate a recommendation for a confirmation test, such as a fingerstick test using a blood sample, according to one embodiment. As shown, meter 200 includes a touchscreen display 205 which illustrates various analyte related data, such as analyte reading 210. Also shown on display 205 is an indicator element 215 for a recommendation for a blood-derived analyte level confirmation. The indicator element 215 shown is a graphical icon of a “blood drop” to represent a recommendation to perform the blood-derived analyte level confirmation.

In certain embodiments, the recommendation to perform a confirmation test may be indicated in other manners with the user interface, alone or in addition to a visual recommendation. For example, the recommendation may be indicated audibly with a speaker or vibrational with a vibrating element. For instance, audible recommendations may include audio text, tones, or other audible sounds.

The recommendation to perform a confirmation test is indicated until one or more predetermined conditions are met, as represented at block 120 of FIG. 1. The predetermined conditions may vary in different embodiments, but should either provide the user with a confirmation that the sensor data is inaccurate, or provide a level of assurance that the sensor data is no longer currently associated with a likelihood of inaccuracy. Furthermore, different predetermined condition may be implemented for different predetermined signal characteristics that are detected, etc.

In certain embodiments, the predetermined conditions may include the completion of a confirmation test by the user. After the completion of the confirmation test, the recommendation to perform the confirmation test is no longer indicated—e.g., the icon removed from the screen. If the confirmation test confirms that the sensor data is sufficiently accurate, then the recommendation to perform the confirmation test is no longer needed and not indicated. If it is confirmed that the sensor data is not sufficiently accurate, then appropriate indications or measures may be initiated, such as communicating the information to the user, disabling of device features such as therapeutic recommendations (e.g., drug administration recommendations such as insulin calculations, etc.), or initiation or deactivation of one or more other measures or features. The recommendation to perform the confirmation test is not indicated (e.g., icon no longer displayed on the display screen) until the next time a predetermined signal characteristic is detected, for example. In one embodiment, a predetermined condition is met when a confirmation test is completed and results in confirmation that the sensor data is sufficiently accurate. If it is confirmed that the sensor data is not sufficiently accurate, then appropriate indications or measures may be initiated, the recommendation to perform a confirmation test may remain indicated with the user interface to recommend another test be performed or to inform the user that the sensor data is inaccurate or potentially inaccurate, for example.

Again, in certain embodiments, the predetermined conditions may include a predetermined period of time—e.g., from the detection of the predetermined signal characteristic, from the time the recommendation is indicated with the user interface, from the time a confirmation test is completed, etc. The predetermined time period may vary in different embodiments. For example, in one embodiment, the predetermined period of time is between 1 and 3 hours since the detection of the predetermined signal characteristic. Furthermore, the predetermined period of time may vary depending on which predetermined signal is detected.

In certain embodiments, a predetermined condition is met when predetermined signal characteristics are no longer detected. For example, after the detection of a predetermined signal characteristic, a recommendation to perform a confirmation test is displayed on the display screen of a user interface. The recommendation is then removed from the display screen when the analyte monitoring system no longer detects a predetermined signal characteristic. The recommendation to perform the confirmation test is then indicated again when a predetermined signal characteristic is detected thereafter, for example.

While in one embodiment the same recommendation is displayed on a display screen and then removed after the predetermined conditions are met, in other embodiments, a different indication may be displayed to indicate that the a recommendation to perform a confirmation test is not required.

The recommendation to perform the confirmation test may be removed at other times in other embodiments. For example, in one embodiment, confirmation by blood glucose test (e.g., either directly performed on the device if a strip port is present, or manually entered blood glucose value by the user or manually entered general confirmation by the user) may remove the confirmation condition. For instance, the blood glucose could confirm that ESA is not present and that the user's blood glucose is in fact low. In such case, subsequent sensor readings may not need to be accompanied by the confirmation request. Similarly, if the confirmation request was due to an algorithm's estimate that perhaps the sensor had become dislodged, evidence (e.g., by confirmation of the reading by the user) may suppress subsequent requests for confirmation permanently or for some period of time.

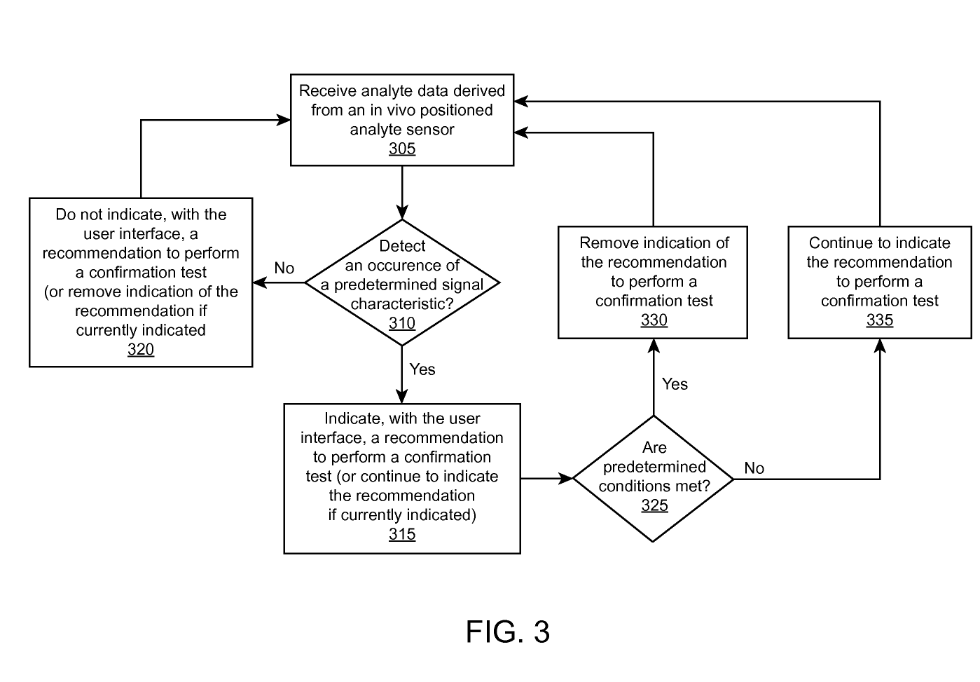

FIG. 3 illustrates an example flowchart for a method of monitoring sensor data and indicating, with a user interface, recommendations to perform confirmation tests, according to one embodiment. For the sake of clarity and brevity, similar features and description as to those described in FIGS. 1-2 are not again repeated here for FIG. 3 but should be understood that they may similarly apply.

At block 305, sensor data that is derived from an in vivo positioned analyte sensor is received. At block 310, it is determined if a predetermined signal characteristic is detected in the sensor data.

If a predetermined signal characteristic is not detected, then a recommendation to perform a confirmation test is not indicated with the user interface, as represented by block 320. Furthermore, in this example embodiment shown, a predetermined condition to remove a recommendation is the absence of any predetermined signal characteristics in the sensor data (e.g., a determination that no predetermined signal characteristics are detected). Thus, if at block 320, a recommendation is already currently being indicated with the user interface—e.g., from a prior detection of a predetermined signal characteristic in the sensor data—then the indication of the recommendation is removed and no longer indicated with the user interface, at block 320, because no predetermined signal characteristic was detected at block 310. In other embodiments where the absence of any predetermined signal characteristics in the sensor data is not a predetermined condition, then if at block 320, a recommendation is already currently being indicated with the user interface—e.g., from a prior detection of a predetermined signal characteristic in the sensor data—then an additional step may be performed (e.g., between block 320 to block 305) to determine if any predetermined conditions are met.

If at block 310, a predetermined signal characteristic is detected, then the recommendation to perform a confirmation test is indicated with the user interface, as represented by block 315. The recommendation may be indicated, for example, by display a corresponding icon on the display screen of the user interface. If a recommendation is already currently being indicated—e.g., from a prior detection of a predetermined signal characteristic in the sensor data—then the recommendation continues to be indicated with the user interface.

At block 325, it is determined whether predetermined conditions are met to remove the recommendation, as represented by block 325. In certain embodiments, the predetermined conditions may include one or more of the following: the completion of a confirmation test, the completion of a confirmation test resulting in a confirmation of accuracy, the elapsing of a predetermined period of time, the absence of any predetermined signal characteristics, etc. If the predetermined conditions are not met, then the recommendation continues to be indicated with the user interfaces, as represented by block 335 and arrow back to 305. If a predetermined condition is met, then the indication of the recommendation is removed and no longer indicated with the user interface, as represented by block 330.

After blocks 320, 330, and 335, the cycle is repeated subsequent sensor data as shown by the arrows returning to block 305. In the embodiment shown, if new sensor data is received and a recommendation is currently being indicated with the user interface (e.g., as represented by the arrow going from block 335 to block 305), then if a predetermined signal characteristic is

[symbol]

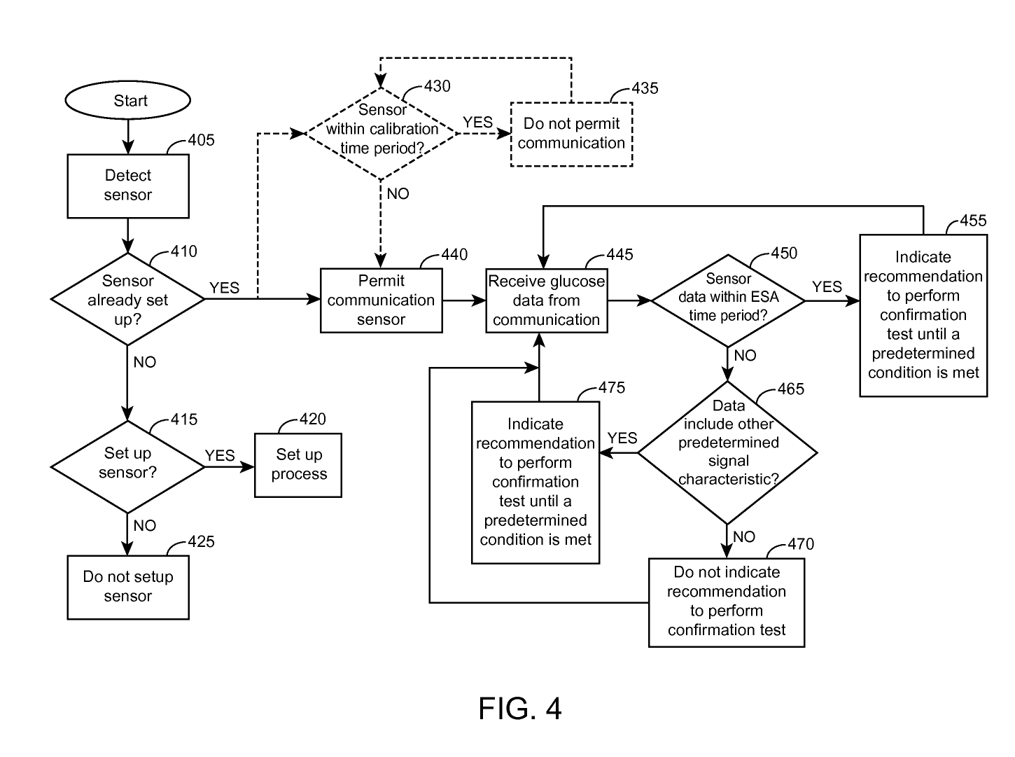

FIG. 4 illustrates a flowchart for a method of monitoring sensor data and indicating, with a user interface, recommendations to perform confirmation tests, according to one embodiment. In FIG. 4, more than one predetermined signal characteristic is implanted, one of which is sensor data that has been acquired during an early sensor attenuation (ESA) time period. As stated previously, the ESA time period may be a predetermined time period associated with the time the analyte sensor was positioned in vivo or otherwise activated. Furthermore, while a glucose monitoring device is described in FIG. 4, other analytes and analyte monitoring devices may be applicable in other embodiments, such as ketone bodies, etc. Again, for the sake of clarity and brevity, similar features and description as to those described in FIGS. 1-3 are not again repeated here for FIG. 4 but should be understood that they may similarly apply.

During operation of the glucose monitoring device (e.g., receiver unit), the device is brought into communication range of a glucose sensor positioned in vivo in the ISF of a test subject. The device detects the sensor, as shown in block 405, and then determined if the sensor is currently set up to work with the device, as shown in block 410.

If the sensor is not currently set up to work with the device, then it is determined if the sensor should be setup to operate with the device, as represented by block 415. For example, the sensor may already be set-up to operate with a different glucose monitoring device and may or may not able to be set-up again with another glucose monitoring device. Furthermore, the glucose monitoring device may already be programmed to work with another sensor and may or may not be able to operate with another sensor. If the glucose monitoring device is able to operate with the sensor, the glucose monitoring device initiates a set-up process, as represented by block 420. In some instances, the user may be prompted to determine if such set-up process should be performed. If the glucose monitoring device is unable to operate with the sensor, then the set-up process is not initiated, as represented by block 425.

If at block 410 it is determined that the sensor is already set up, then the communications between the sensor and receiver are permitted, as represented by block 440. Optionally, in one embodiment, the glucose sensor may require or perform a calibration process during a calibration time period, as represented by dotted blocks 430 and 435. The calibration time period may include a time after sensor insertion that the sensor may not permit sensor-to-receiver communications, or transfer of collected glucose signal data to the device. The calibration period may provide some level of restriction in order to allow the sensor to calibrate or otherwise adjust to insertion within interstitial fluid (ISF), for example. For example, in one embodiment, the sensor may acquire sensor data but the analyte monitoring device does not display or convey any sensor data or recommendations to perform a confirmation test to the user, for example. In another embodiment, the sensor data may not acquire sensor data. Again, in some embodiments, a calibration period may not be implemented.

Blocks 430 and 435 are shown dotted and represent the optional calibration process. From block 410, if the detected sensor is already set up to work with the glucose monitoring device, then it is determined whether the sensor is within a calibration time period. If it determined at block 430 that the sensor is in a calibration time period, then the device is not permitted to communicate with the sensor to receive collected glucose signal data. In some instances, the sensor and device remain in communication range, and the device periodically determines whether the sensor is still in the calibration period, as represented by the arrow returning to block 430.

If at block 430 it is determined that the sensor is not in a calibration time period, then the device is permitted to communicate with the sensor to receive any acquired or collected sensor data, as represented by block 440. At block 445, the sensor transmits acquired or collected glucose signal data to the glucose monitoring device. For example, the sensor may include a transmitter or transceiver to transmit the glucose data to the glucose meter, which includes a wireless receiver or transceiver to receive the data.

At block 450 it is determined if the sensor data is within an ESA time period. The ESA time period may be a predetermined period of time associated with the time of sensor insertion or activation—e.g., 3 days or less, including 12 hours or less, etc. For example, the ESA period of time may be 5 hours, 10 hours, 24 hours, 2 days, etc. The beginning of the ESA period of time may begin at the time of the insertion of the in vivo positioned sensor into the test subject, or another time associated with the sensor insertion—e.g., after activation of the sensor, after a calibration period immediately following the implantation of the sensor, or after some time based on an empirically observed time course of ESA such as associated with typical wound response time.

If the sensor data is determined to be within the ESA time period, then a recommendation for a confirmation test is indicated with the user interface until a predetermined condition is met, as represented by block 455. As long as sensor data continues to be from within the ESA time period, the recommendation remains indicated on the display. The indicating of the recommendation is used broadly herein and may include substantially or generally indicating the recommendation such as to bring it to the attention of the user. For example, the indicating of the recommendation may include flashing or otherwise repeatedly indicating the recommendation on the display of the display screen. As mentioned previously, the device may be programmed for various degrees of restriction until a confirmation test is performed by the user.

If the sensor data is determined to be “outside” the ESA time period, then it is determined whether the analyte signal data includes a predetermined signal characteristic, as represented by block 465. If no predetermined signal characteristic is detected, then a recommendation for a confirmation test is not indicated (or removed if a recommendation is currently indicated) with the user interface of the device, as represented by 470. The process will then be repeated for future communications of sensor data, as represented by the arrow back to block 445. If at block 465 the sensor data does include a predetermined signal characteristic, then a recommendation for a confirmation test will be indicated with the user interface of the device (or remain indicated with the user interface if a recommendation is currently indicated) until a predetermined condition is met, as represented by block 475. The process is then repeated for future communications of sensor data, as represented by the arrow returning to block 445.

Different methods may be used to determine if the sensor data is “within” the ESA time period, or “outside” the ESA time period. For example, the sensor data may be determined to be within or outside the ESA time period based on the time the sensor data was acquired. For example, the sensor data that is received by the analyte monitoring device may include a time stamp or time logging information which may be used to determine the corresponding time period of the collected analyte signal data.

In some instances, sensor data is acquired by the in vivo positioned sensor during a time period that is entirely within the ESA time period, and is accordingly determined to be “within” the ESA time period. Similarly, sensor data acquired by the in vivo positioned sensor during a time period that is entirely after the ESA time period is accordingly determined to be “outside” the ESA time period. In some instances, sensor data may be collected by the in vivo positioned sensor during a time period that encompasses both the ESA time period, or portion thereof, and time after the ESA time period. In such case, determining whether the sensor data is “within” or “outside” the ESA time period may vary in different embodiments. For example, in one embodiment, it may be predetermined that such sensor data is considered to be “within” the ESA time period since it still includes data collected from the ESA time period. In another embodiment, it may be predetermined that such sensor data is considered to be “outside” the ESA time period since it includes data collected after the ESA time period.

In certain embodiments, other factors may be used in determining whether the sensor data is “within” or “outside” the ESA time period. For example, one factor may include a time limit or size limit for the collected data. The time limit or size may be selected, for example, based on the amount or frequency in which the sensor data is collected and transmitted. For instance, the sensor data may be collected and transmitted every five minutes, and if more than 2 minutes of the collected data is during the ESA time period, then the data is considered to be within the ESA time period. As another example, a percentage may be implemented—e.g., if more than 20% of the collected data is during the ESA time period than the data is considered to be “within” the ESA time period.

As yet another example, if the length of time of the sensor data collected during the ESA time period exceeds a threshold percentage of the length of time of the entire ESA time period, then the sensor data collected is considered to be “within” the ESA time period. For instance, if the ESA time period is 12 hours from insertion of the in vivo positioned sensor and the threshold percentage is 25%, then if the analyte signal data includes 3 or more hours of data collected during the ESA time period, it is considered to be “within” the ESA time period. Thus, in this example, if the sensor data consists of data collected during the 9^{th}, 10th, 11^{th}, 12^{th}, and 13th hour after insertion of the in vivo positioned sensor, then it includes 4 hours (9^{th}, 10^{th}, 11^{th}, and 12^{th}) of data collected during the ESA time period and 1 hour (13^{th}) of data collected after the ESA time period. The 4 hours exceeds the threshold of 25% of the ESA time period of 12 hours, and thus the sensor data is considered to be “within” the ESA time period. Similarly, if the sensor data includes less than the 3 hours of data collected during the ESA time period (i.e., less than the 25% threshold), then it will be “outside” the ESA time period. Thus, in this example, if the sensor data consists of data collected during the 11^{th} through 15^{th} hours after insertion of the in vivo positioned sensor, then it includes two hours (11^{th} and 12^{th}) of data collected during the ESA time period and three hours (13^{th}, 14^{th}, and 15^{th}) of data collected after the ESA time period. The 2 hours does not exceed the threshold of 25% of the ESA time period of 12 hours, and thus the sensor data is considered to be “outside” the ESA time period.

FIG. 5A illustrates a plot of an example sensor data collected from an analyte sensor over time, according to one embodiment. Plot 500 includes a plot of the sensor data 501 with analyte level represented along the vertical axis 505 and time represented along the horizontal axis 510. The sensor data 501 includes signal irregularities 515 and 520. In the graph shown, time t₀ represents the time that the sensor is positioned in vivo in the test subject. Time t_{ESA} represents the end of the ESA time period. For example, the ESA period of time begins at time t₀ (e.g., the time of sensor insertion or activation) and ends at time t_{ESA} (e.g., 12 hours after sensor insertion or activation, or 24 hours after sensor insertion or activation, or other predetermined period of time after sensor insertion or activation). The beginning of the ESA period of time not necessarily start with the time of insertion of the sensor into the test subject other embodiments. For example, in one embodiment, the beginning of the ESA period of time may begin at the end of a calibration period t₁, in which case it is not necessarily at the time of insertion of the sensor, but still associated with the time of insertion of the sensor. In another embodiment, the beginning of the ESA period of time may begin at some time after sensor insertion or activation based on an empirically observed time course of ESA such as associated with typical wound response time, and end at the end of the ESA period—e.g., beginning 6 hours after insertion or activation and ending 24 hours after sensor insertion or activation, or beginning 10 hours after sensor insertion or activation and ending 24 hours after sensor insertion or activation, etc.

As shown, signal irregularity 515 includes two data samples that form an outlier in the sensor data, and signal irregularity 520 includes one data sample that forms the dropout in the sensor data. In some instances, an outlier or dropout may have a maximum sample size—e.g., 10 samples, 5 samples, 2 samples, 1 sample, etc.

The plot of sensor data shown represents the cumulative sensor data for a test subject over time, such as that which is collected by a CGM system for instance. For example, sensor data 541 represents an example sensor data that is collected by the in vivo positioned sensor and received by the analyte monitoring device during a communication. The sensor data 541 is collected by the sensor at a time within the ESA time period, and thus the sensor data 541 is considered to be “within” the ESA time period. The sensor data 542 is collected by the sensor at a time after the ESA time period, and thus the sensor data 541 is considered to be “outside” the ESA time period. Sensor data 543 is collected by the sensor at a time encompassing within and after the ESA time period, and thus the sensor data 543 may be considered to be “within” or “outside” the ESA time period depending on the specific definitions implemented—e.g., as similarly above.

FIG. 5B illustrates a plot of sensor data and corresponding slopes at various points in the sensor data, according to one embodiment. As shown, plot 500 includes a plot of sensor data 501 with analyte level represented along the vertical axis 505 and time represented along the horizontal axis 510. Sensor data 501 is show including signal irregularity 582. Several calculated slopes are shown. For example, slopes 550, 555, and 560 are shown at a peak curvature of the sensor data. As shown, the slope of the sensor data gradual changes from the positive slope at slope 550 to zero slope at slope 555 at the top of the peak, and then to a negative slope at slope 560. While only three slope calculations are shown, additional slope calculations may be performed to determine the incremental change in slope, signifying continuous sensor data. Slopes 570 and 575 illustrate slope determinations taken around a “valley” curvature in the signal data. As illustrated, the data point at 582 is an outlier that creates a change in slope that is not gradual or consistent with the flow of the sensor data at the valley—e.g., as shown by slop 575 to slope 580.

The slope determinations may be used in detecting predetermined signal characteristics, such as high rates of changes in analyte values, signal irregularities, etc. For example, an algorithm may use the non-gradual or inconsistent change in slope in identifying the outlier 582. As shown in FIG. 5B, the slopes 580 and 585 generated by outlier 582 are non-gradual and inconsistent with respect to slopes 570,575 and slopes 590,595, respectively.

Furthermore, the outlier defines a large positive slope (slope 580) immediately followed by a large negative slope 585, thus indicating the spike created by the outlier 582. An outlier or dropout below the sensor data signal may create the opposite slope pattern in some instances—e.g., a large negative slope immediately followed by a large positive slope—e.g., 5 mg/dL per minute or greater. An algorithm may use these patterns of slope to identify outliers and dropouts in the sensor data signal. The magnitude jump and/or the span of time between the two slopes may vary depending on the tolerance and definitions implemented for predetermined signal characteristics. The algorithms described above are exemplary and that that any suitable algorithms and method may be used to identify a predetermined signal characteristic. For example, algorithms may identify predetermined signal characteristic (e.g., signal irregularities) using threshold percentage changes in analyte signal value over time, and/or threshold slope changes or deviations, and/or patterns in slope, or any combination thereof. For example, a signal irregularity may be defined by a 30% jump or greater (e.g., 50% jump or greater, or other percentage jump) in analyte level between subsequent values over a relatively short period of time, such as fifteen minutes or less (e.g., five minutes or less, or two minutes or less, or 1 minute). Again, other algorithms may be implemented in another embodiment to identify predetermined signal characteristics.

### Devices and Systems

Embodiments of the present disclosure relate to the continuous in vivo monitoring of the level of one or more analytes using an analyte monitoring device or system. The analyte monitoring device or system may receive sensor data periodically or at various times from sensor electronics coupled to an in vivo positioned sensor to provide such monitoring. The system may include an analyte sensor at least a portion of which is to be positioned beneath a skin surface of a user for a period of time. Systems may include wholly implantable analyte sensors and analyte sensors in which only a portion of the sensor is positioned under the skin and a portion of the sensor resides above the skin, e.g., for contact to a sensor control unit (which may include a transmitter), a receiver/display unit, transceiver, processor, etc. The sensor may be, for example, positionable in vivo in a patient for the continuous monitoring of a level of an analyte in the patient's interstitial fluid and periodically communicated to an analyte monitoring device (e.g., receiver unit).

An analyte sensor may be positioned in contact with interstitial fluid to detect the level of glucose, which detected glucose may be used to infer the glucose level in the user's bloodstream.

Embodiments of the analyte sensors may be configured for monitoring the level of the analyte over a time period which may range from seconds, minutes, hours, days, weeks, to months, or longer. In one embodiment, the analyte sensors, such as glucose sensors, are capable of in vivo detection of an analyte for one hour or more, e.g., a few hours or more, e.g., a few days or more, e.g., three or more days, e.g., five days or more, e.g., seven days or more, e.g., several weeks or more, or one month or more.

As demonstrated herein, the methods of the present disclosure are useful in connection with a device that is used to measure or monitor an analyte (e.g., glucose), such as any such device described herein. These methods may also be used in connection with a device that is used to measure or monitor another analyte (e.g., ketones, ketone bodies, HbA1c, and the like), including oxygen, carbon dioxide, proteins, drugs, or another moiety of interest, for example, or any combination thereof, found in bodily fluid, including subcutaneous fluid, dermal fluid (sweat, tears, and the like), interstitial fluid, or other bodily fluid of interest, for example, or any combination thereof.

FIG. 6 shows an analyte (e.g., glucose) monitoring system, according to one embodiment. Aspects of the subject disclosure are further described primarily with respect to glucose monitoring devices and systems, and methods of glucose detection, for convenience only and such description is in no way intended to limit the scope of the embodiments. It is to be understood that the analyte monitoring system may be configured to monitor a variety of analytes at the same time or at different times.

Analytes that may be monitored include, but are not limited to, acetyl choline, amylase, bilirubin, cholesterol, chorionic gonadotropin, glycosylated hemoglobin (HbA1c), creatine kinase (e.g., CK-MB), creatine, creatinine, DNA, fructosamine, glucose, glucose derivatives, glutamine, growth hormones, hormones, ketones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In embodiments that monitor more than one analyte, the analytes may be monitored at the same or different times.

The analyte monitoring system 600 includes an analyte sensor 601, a data processing unit 602 connectable to the sensor 601, and a primary receiver unit 604. In some instances, the primary receiver unit 604 is configured to communicate with the data processing unit 602 via a communication link 603. In one embodiment, the primary receiver unit 604 may be further configured to transmit data to a data processing terminal 605 to evaluate or otherwise process or format data received by the primary receiver unit 604. The data processing terminal 605 may be configured to receive data directly from the data processing unit 602 via a communication link 607, which may optionally be configured for bi-directional communication. Further, the data processing unit 602 may include a transmitter or a transceiver to transmit and/or receive data to and/or from the primary receiver unit 604 and/or the data processing terminal 605 and/or optionally a secondary receiver unit 606.

Also shown in FIG. 6 is an optional secondary receiver unit 606 which is operatively coupled to the communication link 603 and configured to receive data transmitted from the data processing unit 602. The secondary receiver unit 606 may be configured to communicate with the primary receiver unit 604, as well as the data processing terminal 605. In one embodiment, the secondary receiver unit 606 may be configured for bi-directional wireless communication with each of the primary receiver unit 604 and the data processing terminal 605. As discussed in further detail below, in some instances, the secondary receiver unit 606 may be a de-featured receiver as compared to the primary receiver unit 604, for instance, the secondary receiver unit 606 may include a limited or minimal number of functions and features as compared with the primary receiver unit 604. As such, the secondary receiver unit 606 may include a smaller (in one or more, including all, dimensions), compact housing or embodied in a device including a wrist watch, arm band, PDA, mp3 player, cell phone, etc., for example. Alternatively, the secondary receiver unit 106 may be configured with the same or substantially similar functions and features as the primary receiver unit 604. The secondary receiver unit 606 may include a docking portion configured to mate with a docking cradle unit for placement by, e.g., the bedside for night time monitoring, and/or a bi-directional communication device. A docking cradle may recharge a power supply.

Only one analyte sensor 601, data processing unit 602 and data processing terminal 605 are shown in the embodiment of the analyte monitoring system 600 illustrated in FIG. 6. However, it will be appreciated by one of ordinary skill in the art that the analyte monitoring system 600 may include more than one sensor 601 and/or more than one data processing unit 602, and/or more than one data processing terminal 605. Multiple sensors may be positioned in a user for analyte monitoring at the same or different times.

The analyte monitoring system 600 may be a continuous monitoring system, or semi-continuous, or a discrete monitoring system. In a multi-component environment, each component may be configured to be uniquely identified by one or more of the other components in the system so that communication conflict may be readily resolved between the various components within the analyte monitoring system 600. For example, unique IDs, communication channels, and the like, may be used.

In one embodiment, the sensor 601 is physically positioned in or on the body of a user whose analyte level is being monitored. The sensor 601 may be configured to at least periodically sample the analyte level of the user and convert the sampled analyte level into a corresponding signal for transmission by the data processing unit 602. The data processing unit 602 is coupleable to the sensor 601 so that both devices are positioned in or on the user's body, with at least a portion of the analyte sensor 601 positioned in vivo. The data processing unit may include a fixation element, such as an adhesive or the like, to secure it to the user's body. A mount (not shown) attachable to the user and mateable with the data processing unit 602 may be used. For example, a mount may include an adhesive surface. The data processing unit 602 performs data processing functions, where such functions may include, but are not limited to, filtering and encoding of data signals, each of which corresponds to a sampled analyte level of the user, for transmission to the primary receiver unit 604 via the communication link 603. In one embodiment, the sensor 601 or the data processing unit 602 or a combined sensor/data processing unit may be wholly implantable under the skin surface of the user.

In one embodiment, the primary receiver unit 604 may include an analog interface section including an RF receiver and an antenna that is configured to communicate with the data processing unit 602 via the communication link 603, and a data processing section for processing the received data from the data processing unit 602 including data decoding, error detection and correction, data clock generation, data bit recovery, etc., or any combination thereof.

The primary receiver unit 604 in one embodiment is configured to synchronize with the data processing unit 602 to uniquely identify the data processing unit 602, based on, for example, an identification information of the data processing unit 602, and thereafter, to periodically receive signals transmitted from the data processing unit 602 associated with the monitored analyte levels detected by the sensor 601.

The data processing terminal 605 may include a personal computer, a portable computer including a laptop or a handheld device (e.g., a personal digital assistant (PDA), a telephone including a cellular phone (e.g., a multimedia and Internet-enabled mobile phone including an iPhone™, a Blackberry®, or similar phone), an mp3 player (e.g., an iPOD™, etc.), a pager, and the like), and/or a drug delivery device (e.g., an infusion device), each of which may be configured for data communication with the receiver via a wired or a wireless connection. Additionally, the data processing terminal 605 may further be connected to a data network (not shown) for storing, retrieving, updating, and/or analyzing data corresponding to the detected analyte level of the user.

The data processing terminal 605 may include a drug delivery device (e.g., an infusion device) such as an insulin infusion pump or the like, which may be configured to administer a drug (e.g., insulin) to the user, and which may be configured to communicate with the primary receiver unit 604 for receiving, among others, the measured analyte level. Alternatively, the primary receiver unit 604 may be configured to integrate an infusion device therein so that the primary receiver unit 604 is configured to administer an appropriate drug (e.g., insulin) to users, for example, for administering and modifying basal profiles, as well as for determining appropriate boluses for administration based on, among others, the detected analyte levels received from the data processing unit 602. An infusion device may be an external device or an internal device, such as a device wholly implantable in a user.

In one embodiment, the data processing terminal 605, which may include an infusion device, e.g., an insulin pump, may be configured to receive the analyte signals from the data processing unit 602, and thus, incorporate the functions of the primary receiver unit 604 including data processing for managing the user's insulin therapy and analyte monitoring. In one embodiment, the communication link 603, as well as one or more of the other communication interfaces shown in FIG. 6, may use one or more wireless communication protocols, such as, but not limited to: an RF communication protocol, an infrared communication protocol, a Bluetooth enabled communication protocol, an 802.11x wireless communication protocol, or an equivalent wireless communication protocol which would allow secure, wireless communication of several units (for example, per Health Insurance Portability and Accountability Act (HIPPA) requirements), while avoiding potential data collision and interference.

FIG. 7 is a block diagram of the data processing unit 602 shown in FIG. 6 in accordance with one embodiment. Data processing unit 602 includes an analog interface 701 configured to communicate with the sensor 601, a user input 702, and a temperature measurement section 703, each of which is operatively coupled to processor 704 such as a central processing unit (CPU). Furthermore, unit 602 is shown to include a serial communication section 705, clock 708, and an RF transmitter 706, each of which is also operatively coupled to the processor 704. Moreover, a power supply 707 such as a battery is also provided in unit 602 to provide the necessary power.

In other embodiments, the data processing unit may not include all components in the exemplary embodiment shown. User input and/or interface components may be included or a data processing unit may be free of user input and/or interface components. In one embodiment, one or more application-specific integrated circuits (ASIC) may be used to implement one or more functions or routines associated with the operations of the data processing unit (and/or receiver unit) using for example one or more state machines and buffers.

The analyte sensor 601 is shown including four contacts, three of which are electrodes: a work electrode (W) 710, a reference electrode (R) 712, and a counter electrode (C) 713, each operatively coupled to the analog interface 701 of the data processing unit 602. This embodiment also shows an optional guard contact (G) 711. Fewer or greater electrodes may be employed. For example, the counter and reference electrode functions may be served by a single counter/reference electrode. In some cases, there may be more than one working electrode and/or reference electrode and/or counter electrode, etc.

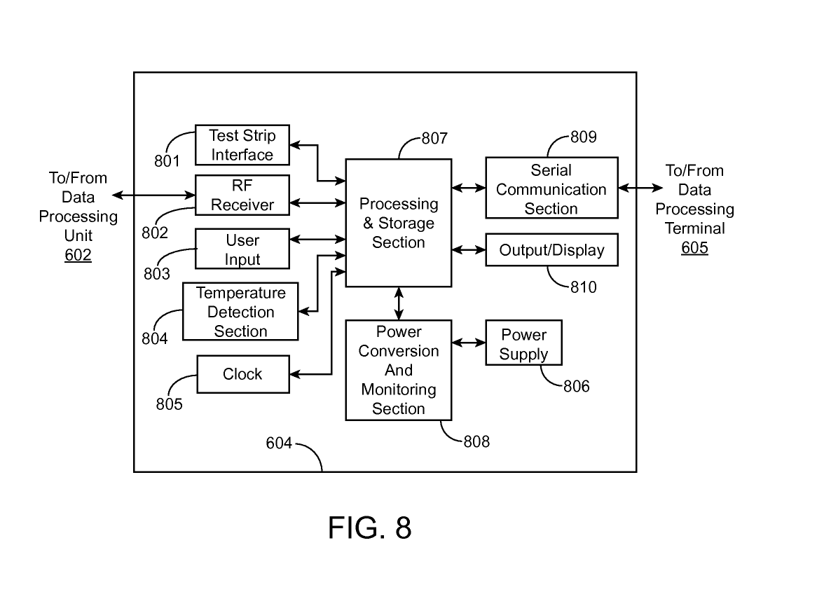

FIG. 8 is a block diagram of an embodiment of a receiver/monitor unit such as the primary receiver unit 604 of the analyte monitoring system shown in FIG. 6. The primary receiver unit 604 includes one or more of: a test strip interface 801, an RF receiver 802, a user input 803, an optional temperature detection section 804, and a clock 805, each of which is operatively coupled to a processing and storage section 807. The primary receiver unit 604 also includes a power supply 806 operatively coupled to a power conversion and monitoring section 808. Further, the power conversion and monitoring section 808 is also coupled to the processing and storage section 807. Moreover, also shown are a receiver serial communication section 809, and an output 810, each operatively coupled to the processing and storage section 807. The primary receiver unit 604 may include user input and/or interface components or may be free of user input and/or interface components.

In one embodiment, the test strip interface 801 includes an analyte testing portion (e.g., a glucose level testing portion) to receive a blood (or other body fluid sample) analyte test or information related thereto. For example, the test strip interface 801 may include a test strip port to receive a test strip (e.g., a glucose test strip). The device may determine the analyte level of the test strip, and optionally display (or otherwise notice) the analyte level on the output 810 of the primary receiver unit 604. Any suitable test strip may be employed, e.g., test strips that only require a very small amount (e.g., 3 microliters or less, e.g., 1 microliter or less, e.g., 0.5 microliters or less, e.g., 0.1 microliters or less), of applied sample to the strip in order to obtain accurate glucose information. Embodiments of test strips include, e.g., Freestyle® blood glucose test strips from Abbott Diabetes Care, Inc. (Alameda, Calif.). Glucose information obtained by an in vitro glucose testing device may be used for a variety of purposes, computations, etc. For example, the information may be used to calibrate sensor 601, confirm results of sensor 601 to increase the confidence thereof (e.g., in instances in which information obtained by sensor 601 is employed in therapy related decisions), etc.

In further embodiments, the data processing unit 602 and/or the primary receiver unit 604 and/or the secondary receiver unit 606, and/or the data processing terminal/infusion device 605 may be configured to receive the analyte value wirelessly over a communication link from, for example, a blood glucose meter. In further embodiments, a user manipulating or using the analyte monitoring system 600 (FIG. 6) may manually input the analyte value using, for example, a user interface (for example, a keyboard, keypad, voice commands, and the like) incorporated in one or more of the data processing unit 602, the primary receiver unit 604, secondary receiver unit 606, or the data processing terminal/infusion device 605.

Additional detailed descriptions are provided in U.S. Pat. Nos. 5,262,035; 5,264,104; 5,262,305; 5,320,715; 5,593,852; 6,175,752; 6,650,471; 6,746, 582, and 7,811,231, each of which is incorporated herein by reference in their entirety.

In some instances, the analyte monitoring device includes processing circuitry that is able to determine a level of the analyte and activate an alarm system if the analyte level exceeds a threshold. The analyte monitoring device, in these embodiments, has an alarm system and may also include a display, such as an LCD or LED display. An alarm may also be activated if the sensor readings indicate a value that is beyond a measurement range of the sensor. For glucose, the physiologically relevant measurement range may be 30-400 mg/dL, including 40-300 mg/dL and 50-250 mg/dL, of glucose in the interstitial fluid. The alarm system may also, or alternatively, be activated when the rate-of-change or acceleration of the rate-of-change in analyte level increase or decrease reaches or exceeds a threshold rate or acceleration. For example, in the case of an in vivo based glucose monitor, the alarm system might be activated if the rate-of-change in glucose concentration exceeds a threshold value which might indicate that a hyperglycemic or hypoglycemic condition is likely to occur. A system may also include system alarms that notify a user of system information such as battery condition, calibration, sensor dislodgment, sensor malfunction, etc. Alarms may be, for example, auditory and/or visual. Other sensory-stimulating alarm systems may be used including alarm systems which heat, cool, vibrate, or produce a mild electrical shock when activated.

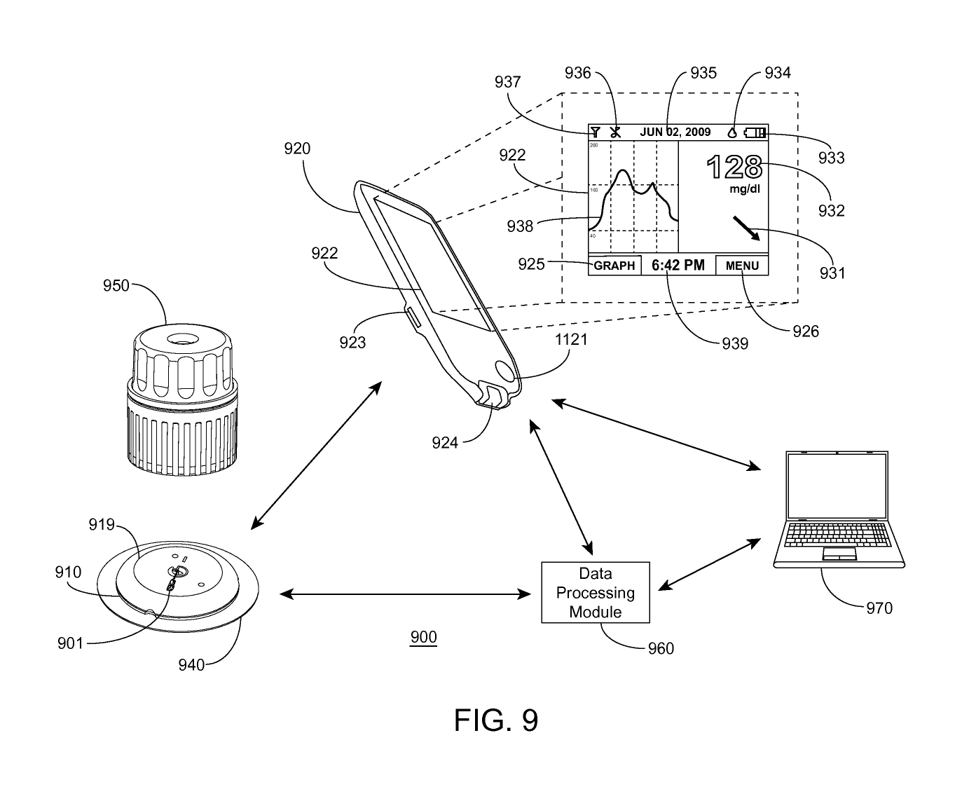

FIG. 9 illustrates another example analyte (e.g., glucose) monitoring system, according to one embodiment. FIG. 9 shows an exemplary in vivo-based analyte monitoring system 900 in accordance with embodiments of the present disclosure. As shown, in certain embodiments, analyte monitoring system 900 includes on body electronics 910 electrically coupled to in vivo analyte sensor 901 (a proximal portion of which is shown in FIG. 9) and attached to adhesive layer 940 for attachment on a skin surface on the body of a user. On body electronics 910 includes on body housing 919, that defines an interior compartment. Also shown in FIG. 9 is insertion device 950 that, when operated, positions a portion of analyte sensor 901 in vivo through a skin surface and in fluid contact with ISF, and positions on body electronics 910 and adhesive layer 940 on a skin surface In certain embodiments, on body electronics 910, analyte sensor 901 and adhesive layer 940 are sealed within the housing of insertion device 950 before use, and in certain embodiments, adhesive layer 940 is also sealed within the housing or itself provides a terminal seal of the insertion device 950. Devices, systems and methods that may be used with embodiments herein are described, e.g., in U.S. patent application Ser. No. 12/698,129 and U.S. Provisional Application Nos. 61/238,646, 61/246,825, 61/247,516, 61/249,535, 61/317,243, 61/345,562, and 61/361,374, the disclosures of each of which are incorporated herein by reference for all purposes.

Referring back to the FIG. 9, analyte monitoring system 100 includes display device 920 which includes a display 922 to output information to the user, an input component 921 such as a button, actuator, a touch sensitive switch, a capacitive switch, pressure sensitive switch, jog wheel or the like, to input data or command to display device 920 or otherwise control the operation of display device 920. Furthermore, the indicator element for a recommendation for a blood-derived confirmation test is displayed on display 922. Embodiments will be described herein as display devices for exemplary purposes which are in no way intended to limit the embodiments of the present disclosure. It will be apparent that a displayless device may also be used in certain embodiments—e.g., where the recommendation for a blood-derived confirmation test is provided audibly.

In certain embodiments, display 922 and input component 921 may be integrated into a single component, for example a display that can detect the presence and location of a physical contact touch upon the display such as a touch screen user interface. In such embodiments, the user may control the operation of display device 920 by utilizing a set of pre-programmed motion commands, including, but not limited to, single or double tapping the display, dragging a finger or instrument across the display, motioning multiple fingers or instruments toward one another, motioning multiple fingers or instruments away from one another, etc. In certain embodiments, a display includes a touch screen having areas of pixels with single or dual function capacitive elements that serve as LCD elements and touch sensors.

Display device 920 also includes data communication port 923 for wired data communication with external devices such as remote terminal (personal computer) 970, for example. Example embodiments of the data communication port 923 include USB port, mini USB port, RS-232 port, Ethernet port, Firewire port, or other similar data communication ports configured to connect to the compatible data cables. Display device 920 may also include an integrated in vitro glucose meter, including in vitro test strip port 924 to receive an in vitro glucose test strip for performing in vitro blood glucose measurements.

Referring still to FIG. 9, display 922 in certain embodiments is configured to display a variety of information—some or all of which may be displayed at the same or different time on display 922. Display 922 may include but is not limited to graphical display 938, for example, providing a graphical output of glucose values over a monitored time period (which may show important markers such as meals, exercise, sleep, heart rate, blood pressure, etc., numerical display 932, for example, providing monitored glucose values (acquired or received in response to the request for the information), and trend or directional arrow display 931 that indicates a rate of analyte change and/or a rate of the rate of analyte change, e.g., by moving locations on display 922. Again, the indicator element for a recommendation for a blood-derived confirmation test is displayed on display 922.

As further shown in FIG. 9, display 922 may also include date display 935 providing for example, date information for the user, time of day information display 939 providing time of day information to the user, battery level indicator display 933 which graphically shows the condition of the battery (rechargeable or disposable) of the display device 920, sensor calibration status icon display 934 for example, in monitoring systems that require periodic, routine or a predetermined number of user calibration events, notifying the user that the analyte sensor calibration is necessary, audio/vibratory settings icon display 936 for displaying the status of the audio/vibratory output or alarm state, and wireless connectivity status icon display 937 that provides indication of wireless communication connection with other devices such as on body electronics, data processing module 960, and/or remote terminal 970. As additionally shown in FIG. 9, display 922 may further include simulated touch screen buttons 925,926 for accessing menus, changing display graph output configurations or otherwise for controlling the operation of display device 920.

Referring back to FIG. 9, in certain embodiments, display 922 of display device 920 may be additionally, or instead of visual display, configured to output alarms notifications such as alarm and/or alert notifications, glucose values etc., which may be audible, tactile, or any combination thereof. In one aspect, the display device 920 may include other output components such as a speaker, vibratory output component and the like to provide audible and/or vibratory output indication to the user in addition to the visual output indication provided on display 922. Further details and other display embodiments can be found in, e.g., U.S. patent application Ser. No. 12/871,901, U.S. provisional application Nos. 61/238,672, 61/247,541, 61/297,625, the disclosures of each of which are incorporated herein by reference for all purposes.

After the positioning of on body electronics 910 on the skin surface and analyte sensor 901 in vivo to establish fluid contact with ISF (or other appropriate body fluid), on body electronics 910 in certain embodiments is configured to wirelessly communicate analyte related data (such as, for example, data corresponding to monitored analyte level and/or monitored temperature data, and/or stored historical analyte related data) when on body electronics 910 receives a command or request signal from display device 120. In certain embodiments, on body electronics 910 may be configured to at least periodically broadcast real time data associated with monitored analyte level which is received by display device 920 when display device 920 is within communication range of the data broadcast from on body electronics 910, i.e., it does not need a command or request from a display device to send information.

For example, display device 920 may be configured to transmit one or more commands to on body electronics 910 to initiate data transfer, and in response, on body electronics 910 may be configured to wirelessly transmit stored analyte related data collected during the monitoring time period to display device 920. Display device 920 may in turn be connected to a remote terminal 970 such as a personal computer and functions as a data conduit to transfer the stored analyte level information from the on body electronics 910 to remote terminal 970. In certain embodiments, the received data from the on body electronics 910 may be stored (permanently or temporarily) in one or more memory of the display device 920. In certain other embodiments, display device 920 is configured as a data conduit to pass the data received from on body electronics 910 to remote terminal 970 that is connected to display device 920.

Referring still to FIG. 9, also shown in analyte monitoring system 900 are data processing module 960 and remote terminal 970. Remote terminal 970 may include a personal computer, a server terminal a laptop computer or other suitable data processing devices including software for data management and analysis and communication with the components in the analyte monitoring system 900. For example, remote terminal 970 may be connected to a local area network (LAN), a wide area network (WAN), or other data network for uni-directional or bi-directional data communication between remote terminal 970 and display device 920 and/or data processing module 960.

Remote terminal 970 in certain embodiments may include one or more computer terminals located at a physician's office or a hospital. For example, remote terminal 970 may be located at a location other than the location of display device 920. Remote terminal 970 and display device 920 could be in different rooms or different buildings. Remote terminal 970 and display device 920 could be at least about one mile apart, e.g., at least about 110 miles apart, e.g., at least about 1100 miles apart. For example, remote terminal 970 could be in the same city as display device 920, remote terminal 970 could be in a different city than display device 920, remote terminal 970 could be in the same state as display device 920, remote terminal 970 could be in a different state than display device 920, remote terminal 970 could be in the same country as display device 920, or remote terminal 970 could be in a different country than display device 920, for example. In certain embodiments, a separate, optional data communication/processing device such as data processing module 960 may be provided in analyte monitoring system 900. Data processing module 160 may include components to communicate using one or more wireless communication protocols such as, for example, but not limited to, infrared (IR) protocol, Bluetooth protocol, Zigbee protocol, and 802.11 wireless LAN protocol. Additional description of communication protocols including those based on Bluetooth protocol and/or Zigbee protocol can be found in U.S. Patent Publication No. 2006/0193375 incorporated herein by reference for all purposes. Data processing module 960 may further include communication ports, drivers or connectors to establish wired communication with one or more of display device 920, on body electronics 910, or remote terminal 970 including, for example, but not limited to USB connector and/or USB port, Ethernet connector and/or port, FireWire connector and/or port, or RS-232 port and/or connector.

In certain embodiments, control logic or microprocessors of on body electronics 910 include software programs to determine future or anticipated analyte levels based on information obtained from analyte sensor 901, e.g., the current analyte level, the rate of change of the analyte level, the acceleration of the analyte level change, and/or analyte trend information determined based on stored monitored sensor data providing a historical trend or direction of analyte level fluctuation as function time during monitored time period. Predictive alarm parameters may be programmed or programmable in display device 920, or the on body electronics 910, or both, and output to the user in advance of anticipating the user's analyte level reaching the future level. This provides the user an opportunity to take timely corrective action.

Information, such as variation or fluctuation of the monitored analyte level as a function of time over the monitored time period providing analyte trend information, for example, may be determined by one or more control logic or microprocessors of display device 920, data processing module 160, and/or remote terminal 970, and/or on body electronics 910. Such information may be displayed as, for example, a graph (such as a line graph) to indicate to the user the current and/or historical and/or and predicted future analyte levels as measured and predicted by the analyte monitoring system 900. Such information may also be displayed as directional arrows (for example, see trend or directional arrow display 931) or other icon(s), e.g., the position of which on the screen relative to a reference point indicated whether the analyte level is increasing or decreasing as well as the acceleration or deceleration of the increase or decrease in analyte level. This information may be utilized by the user to determine any necessary corrective actions to ensure the analyte level remains within an acceptable and/or clinically safe range. Other visual indicators, including colors, flashing, fading, etc., as well as audio indicators including a change in pitch, volume, or tone of an audio output and/or vibratory or other tactile indicators may also be incorporated into the display of trend data as means of notifying the user of the current level and/or direction and/or rate of change of the monitored analyte level. For example, based on a determined rate of glucose change, programmed clinically significant glucose threshold levels (e.g., hyperglycemic and/or hypoglycemic levels), and current analyte level derived by an in vivo analyte sensor, the system 900 may include an algorithm stored on computer readable medium to determine the time it will take to reach a clinically significant level and will output notification in advance of reaching the clinically significant level, e.g., 30 minutes before a clinically significant level is anticipated, and/or 20 minutes, and/or 10 minutes, and/or 5 minutes, and/or 3 minutes, and/or 1 minute, and so on, with outputs increasing in intensity or the like.

Referring again back to FIG. 9, in certain embodiments, software algorithm(s) for execution by data processing module 960 may be stored in an external memory device such as an SD card, microSD card, compact flash card, XD card, Memory Stick card, Memory Stick Duo card, or USB memory stick/device including executable programs stored in such devices for execution upon connection to the respective one or more of the on body electronics 910, remote terminal 970 or display device 920. In a further aspect, software algorithms for execution by data processing module 160 may be provided to a communication device such as a mobile telephone including, for example, WiFi or Internet enabled smart phones or personal digital assistants (PDAs) as a downloadable application for execution by the downloading communication device.

Examples of smart phones include Windows®, Android™, iPhone® operating system, Palm® WebOS™, Blackberry® operating system, or Symbian® operating system based mobile telephones with data network connectivity functionality for data communication over an internet connection and/or a local area network (LAN). PDAs as described above include, for example, portable electronic devices including one or more microprocessors and data communication capability with a user interface (e.g., display/output unit and/or input unit, and configured for performing data processing, data upload/download over the internet, for example. In such embodiments, remote terminal 170 may be configured to provide the executable application software to the one or more of the communication devices described above when communication between the remote terminal 970 and the devices are established.

In still further embodiments, executable software applications may be provided over-the-air (OTA) as an OTA download such that wired connection to remote terminal 970 is not necessary. For example, executable applications may be automatically downloaded as software download to the communication device, and depending upon the configuration of the communication device, installed on the device for use automatically, or based on user confirmation or acknowledgement on the communication device to execute the installation of the application. The OTA download and installation of software may include software applications and/or routines that are updates or upgrades to the existing functions or features of data processing module 960 and/or display device 920.

Referring back to remote terminal 970 of FIG. 9, in certain embodiments, new software and/or software updates such as software patches or fixes, firmware updates or software driver upgrades, among others, for display device 920 and/or on body electronics 910 and/or data processing module 960 may be provided by remote terminal 970 when communication between the remote terminal 970 and display device 920 and/or data processing module 960 is established. For example, software upgrades, executable programming changes or modification for on body electronics 910 may be received from remote terminal 970 by one or more of display device 920 or data processing module 960, and thereafter, provided to on body electronics 910 to update its software or programmable functions. For example, in certain embodiments, software received and installed in on body electronics 910 may include software bug fixes, modification to the previously stalled software parameters (modification to analyte related data storage time interval, resetting or adjusting time base or information of on body electronics 910, modification to the transmitted data type, data transmission sequence, or data storage time period, among others). Additional details describing field upgradability of software of portable electronic devices, and data processing are provided in U.S. application Ser. Nos. 12/698,124, 12/794,721, 12/699,653, and 12/699,844, and U.S. Provisional Application Nos. 61,359,265, and 61/325,155 the disclosure of which is incorporated by reference herein for all purposes.

In some aspects, the display device (also referred to herein as “analyte monitoring device” or simply “device”) is configured to receive a signal from a remote sensor using radio-frequency identification (RFID) technology.

This configuration may be used to provide glucose on demand capabilities, for example, in which case when a measurement reading is desired, the analyte monitoring device is brought within close vicinity of the implantable sensor. In other embodiments the wireless communication unit may communicate with the sensor using a different wireless communication technology than RFID. When within range, the device may be configured to verify that the sensor is the appropriate sensor that it has been configured to operate with. If not, the device ignores the sensor and does not initiate operation with the sensor. If so, the device initiates operation with the sensor.

### Drug Delivery System

The present disclosure may also relate to sensors used in sensor-based drug delivery systems. The system may provide a drug to counteract the high or low level of the analyte in response to the signals from one or more sensors. Alternatively, the system may monitor the drug concentration to ensure that the drug remains within a desired therapeutic range. The drug delivery system may include one or more (e.g., two or more) sensors, a processing unit such as a transmitter, a receiver/display unit, and a drug administration system. In some cases, some or all components may be integrated in a single unit. A sensor-based drug delivery system may use data from the one or more sensors to provide necessary input for a control algorithm/mechanism to adjust the administration of drugs, e.g., automatically or semi-automatically. As an example, a glucose sensor may be used to control and adjust the administration of insulin from an external or in vivo positioned insulin pump.

Each of the various references, presentations, publications, provisional and/or non-provisional U.S. Patent Applications, U.S. Patents, non-U.S. Patent Applications, and/or non-U.S. Patents that have been identified herein, is incorporated herein by reference in its entirety.

Other embodiments and modifications within the scope of the present disclosure will be apparent to those skilled in the relevant art. Various modifications, processes, as well as numerous structures to which the embodiments of the present disclosure may be applicable will be readily apparent to those of skill in the art to which the present disclosure is directed upon review of the specification. Various aspects and features of the present disclosure may have been explained or described in relation to understandings, beliefs, theories, underlying assumptions, and/or working or prophetic examples, although it will be understood that the present disclosure is not bound to any particular understanding, belief, theory, underlying assumption, and/or working or prophetic example. Although various aspects and features of the present disclosure may have been described largely with respect to applications, or more specifically, medical applications, involving diabetic humans, it will be understood that such aspects and features also relate to any of a variety of applications involving non-diabetic humans and any and all other animals. Further, the various aspects and features of the present disclosure relate to any variety of in vivo based sensors. Although an aspect and feature of the present disclosure may have been described largely with respect to applications involving partially implanted sensors, such as transcutaneous or subcutaneous sensors, it will be understood that such aspects and features also relate to any of a variety of sensors that are suitable for use in connection with the body of an animal or a human, such as those suitable for use as fully implanted in the body of an animal or a human. Finally, although the various aspects and features of the present disclosure have been described with respect to various embodiments and specific examples herein, all of which may be made or carried out conventionally, it will be understood that the invention is entitled to protection within the full scope of the appended claims.

### Additional Example Embodiments

As stated above, in some aspects of the present disclosure, methods of monitoring sensor data and indicating recommendations for confirmation tests on a user interface are provided. The method includes receiving sensor data over time, wherein the sensor data is derived from an in vivo positioned analyte sensor; detecting, with processing circuitry, an occurrence of a predetermined signal characteristic in the sensor data, wherein the predetermined signal characteristic is associated with a likelihood of inaccuracy of the sensor data; and indicating, on a user interface, a recommendation for a confirmation test after the occurrence of the predetermined signal characteristic is detected.

In certain embodiments, the one or more predetermined signal characteristic includes a time period associated with a time of sensor insertion. In certain embodiments, the one or more predetermined signal characteristics includes high rates of change in the sensor data.

In certain embodiments, the one or more predetermined signal characteristics includes a predetermined magnitude range for the sensor data. In certain embodiments, the predetermined magnitude range is associated with low levels of analyte. In certain embodiments, the one or more predetermined signal characteristics includes exceeding a minimum threshold level of calculated accuracy or confidence in accuracy.

In certain embodiments, the one or more predetermined signal characteristics includes a signal irregularity. In some instances, the signal irregularity includes an occurrence of a spike in the sensor data generated by one or more signal outliers, wherein the spike has an associated rate-of-change of 5 mg/dL per minute or greater. In some instances, the signal irregularity includes an occurrence of a spike in the sensor data generated by one or more dropouts in the sensor data, wherein the spike has an associated rate-of-change of 5 mg/dL per minute or greater. In certain embodiments, the methods include calculating slopes for the sensor data, wherein the signal irregularity is determined based on the calculated slopes.

In certain embodiments, the method includes temporarily disabling one or more features of the analyte monitoring system after the occurrence of the predetermined signal characteristic is detected. In some instances, the one or more features include a therapeutic recommendation based on the sensor data. In certain embodiments, the method further includes receiving an indication of user input acknowledging the recommendation for a confirmation test, and re-enabling the one or more features of the analyte monitoring system. In some instances, the indication of the user input includes an indication of a completion of a confirmation test.

In certain embodiments, the recommendation for the confirmation test is displayed on a display of the user interface.

In certain embodiments, the sensor data is derived from interstitial fluid and the recommendation for a confirmation test is a recommendation for a blood-derived confirmation test. In certain embodiments, the analyte is glucose or a ketone body or both.

In certain embodiments, the methods include removing the indication of the recommendation for the confirmation test after a predetermined condition occurs. In some instances, the methods further include detecting, with the processing circuitry, an occurrence of a subsequent predetermined signal characteristic in subsequent sensor data received, and indicating, with the user interface, a subsequent recommendation for a confirmation test after the subsequent occurrence of the subsequent predetermined signal characteristic is detected.

In certain embodiments, the predetermined condition is a completion of a confirmation test. In certain embodiments, the predetermined condition is a completion of a confirmation test resulting in a confirmation of accuracy. In certain embodiments, the predetermined condition is an absence of any predetermined signal characteristics in the sensor data. In certain embodiments, the predetermined condition is an elapse of a predetermined period of time. In some instances, the predetermined period of time is between 1 and 3 hours after the detection of the predetermined signal characteristic.

In some aspects of the present disclosure, analyte monitoring systems for monitoring sensor data and indicating recommendations for confirmation tests on a user interface are provided. The analyte monitoring systems include a user interface to indicate recommendations for confirmation tests, processing circuitry operably coupled to the user interface, and memory operably coupled to the processing circuitry. The memory includes instructions stored therein, which when executed by the processing circuitry, cause the processing circuitry to receive sensor data over time, wherein the sensor data is derived from an in vivo positioned analyte sensor, detect an occurrence of a predetermined signal characteristic in the sensor data, wherein the predetermined signal characteristic is associated with a likelihood of inaccuracy of the sensor data, and indicate, on a user interface, a recommendation for a confirmation test after the occurrence of the predetermined signal characteristic is detected.

In certain embodiments, the one or more predetermined signal characteristic includes a time period associated with a time of sensor insertion. In certain embodiments, the one or more predetermined signal characteristics includes high rates of change in the sensor data. In certain embodiments, the one or more predetermined signal characteristics includes a predetermined magnitude range for the sensor data. In certain embodiments, the predetermined magnitude range is associated with low levels of analyte. In certain embodiments, the one or more predetermined signal characteristics includes exceeding a minimum threshold level of calculated accuracy or confidence in accuracy.

In certain embodiments, the one or more predetermined signal characteristics includes a signal irregularity. In some instances, the signal irregularity includes an occurrence of a spike in the sensor data generated by one or more signal outliers in the sensor data, wherein the spike has an associated rate-of-change of 5 mg/dL per minute or greater. In some instances, the signal irregularity includes an occurrence of a spike in the sensor data generated by one or more dropouts in the sensor data, wherein the spike has an associated rate-of-change of 5 mg/dL per minute or greater. In certain embodiments, the instructions include instructions, which when executed by the processing circuitry, cause the processing circuitry to calculate slopes for the sensor data, wherein the signal irregularity is determined based on the calculated slopes.

In certain embodiments, the instructions include instructions, which when executed by the processing circuitry, cause the processing circuitry to temporarily disable one or more features of the analyte monitoring system after the occurrence of the predetermined signal characteristic is detected. In some instances, the one or more features include a therapeutic recommendation based on the sensor data. In certain embodiments, the instructions include instructions, which when executed by the processing circuitry, cause the processing circuitry to receive an indication of user input acknowledging the recommendation for a confirmation test, and re-enable the one or more features of the analyte monitoring system. In some instances, the indication of the user input includes an indication of a completion of a confirmation test.

In certain embodiments, the recommendation for the confirmation test is displayed on a display of the user interface.

In certain embodiments, the sensor data is derived from interstitial fluid and the recommendation for a confirmation test is a recommendation for a blood-derived confirmation test.

In certain embodiments, the analyte monitoring system includes a wireless receiver. In some instances, the analyte monitoring device further includes an in vivo positionable analyte sensor configured to operably communicate with the wireless receiver. In some instances, the in vivo positionable analyte sensor includes: a piercing member to penetrate skin of a test subject and contact interstitial fluid, memory to store sensor data collected over time, and a transmitter to transmit the sensor data to the wireless receiver.

In certain embodiments, the analyte is glucose or a ketone body.

In certain embodiments, the instructions include instructions, which when executed by the processing circuitry, cause the processing circuitry to remove the indication of the recommendation for the confirmation test after a predetermined condition occurs. In some instances, the instructions further include instructions, which when executed by the processing circuitry, cause the processing circuitry to detect an occurrence of a subsequent predetermined signal characteristic in subsequent sensor data received, and indicate, with the user interface, a subsequent recommendation for a confirmation test after the subsequent occurrence of the subsequent predetermined signal characteristic is detected.

In certain embodiments, the predetermined condition is a completion of a confirmation test. In certain embodiments, the predetermined condition is a completion of a confirmation test resulting in a confirmation of accuracy. In certain embodiments, the predetermined condition is an absence of any predetermined signal characteristics in the sensor data. In certain embodiments, the predetermined condition is an elapse of a predetermined period of time. In some instances, the predetermined period of time is between 1 and 3 hours after the detection of the predetermined signal characteristic.

In certain embodiments, the processing circuitry is included within an analyte monitoring device that is configured to communicate with an in vivo positioned analyte sensor.

In certain embodiments, the processing circuitry is included within sensor electronics in an in vivo positionable analyte sensor, and the sensor electronics are configured to operably couple to a piercing member penetrating the skin of a test subject and contact interstitial fluid.

In certain embodiments, the processing circuitry includes processing circuitry included on an analyte monitoring device that is configured to communicate with an in vivo positioned analyte sensor, and processing circuitry included within sensor electronics in the in vivo positionable analyte sensor, the sensor electronics configured to operably couple to a piercing member penetrating the skin of a test subject and contacting interstitial fluid, and configured to communicate with the analyte monitoring device. For instance, one or more steps of the methods described herein may be performed by the sensor electronics on the in vivo positioned sensor and the other steps performed by the analyte monitoring device.

It should be understood that techniques introduced above can be implemented by programmable circuitry programmed or configured by software and/or firmware, or they can be implemented entirely by special-purpose “hardwired” circuitry, or in a combination of such forms. Such special-purpose circuitry (if any) can be in the form of, for example, one or more application-specific integrated circuits (ASICS), programmable logic devices (PLDs), field-programmable gate arrays (FPGAs), etc.

Software or firmware implementing the techniques introduced herein may be stored on a machine-readable storage medium (also referred to herein generally as a computer-readable medium or computer-readable storage medium) and may be executed by one or more general-purpose or special-purpose programmable microprocessors. A “machine-readable medium”, as the term is used herein, includes any mechanism that can store information in a form accessible by a machine (a machine may be, for example, a computer, network device, cellular phone, personal digital assistant (PDA), manufacturing took, any device with one or more processors, etc.). For example, a machine-accessible medium includes recordable/non-recordable media (e.g., read-only memory (ROM), random access memory (RAM), magnetic disk storage media, optical storage media, flash memory devices, etc.), etc.

Furthermore, a data processing device or system, such as a computer or computer system may be configured to execute some of the techniques introduced herein. The computer may include, for example, a processing device, memory with instructions stored therein to perform the techniques, input/output device elements (e.g., a monitor, keyboard, etc.), etc. For example, the device or system may be used to configure, calibrate, or otherwise program an analyte monitoring device intended to perform analyte measurements, such as analyte point measurements and/or analyte rate-of-change measurements. In some aspects of the present disclosure, some of the techniques described herein may be provided to the device or system from an article of manufacture including the machine readable medium described above.

The preceding examples are put forth so as to provide those of ordinary skill in the art with a complete disclosure and description of how to make and use the embodiments of the invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

That which is claimed is:

1. A method of monitoring sensor data and indicating recommendations for confirmation tests on a user interface, the method comprising:
receiving sensor data over time, wherein the sensor data is derived from an in vivo positioned analyte sensor;
detecting, with processing circuitry, an occurrence of a predetermined signal characteristic in the sensor data, wherein the predetermined signal characteristic is associated with a likelihood of inaccuracy of the sensor data; and
indicating, on a user interface, a recommendation for a confirmation test after the occurrence of the predetermined signal characteristic is detected.

2. The method of claim 1, wherein the one or more predetermined signal characteristic comprises a time period associated with a time of sensor insertion.

3. The method of claim 1, wherein the one or more predetermined signal characteristics comprises high rates of change in the sensor data.

4. The method of claim 1, wherein the one or more predetermined signal characteristics comprises a predetermined magnitude range for the sensor data.

5. The method of claim 4, wherein the predetermined magnitude range is associated with low levels of analyte.

6. The method of claim 1, wherein the one or more predetermined signal characteristics comprises exceeding a minimum threshold level of calculated accuracy or confidence in accuracy.

7. The method of claim 1, wherein the one or more predetermined signal characteristics comprises a signal irregularity.

8. The method of claim 7, wherein the signal irregularity comprises an occurrence of a spike in the sensor data generated by one or more signal outliers in the sensor data, wherein the spike has an associated rate-of-change of 5 mg/dL per minute or greater.

9. The method of claim 7, wherein the spike is an occurrence of a spike in the sensor data generated by one or more dropouts in the sensor data, wherein the spike has an associated rate-of-change of 5 mg/dL per minute or greater.

10. The method of claim 7, comprising calculating slopes for the sensor data, wherein the signal irregularity is determined based on the calculated slopes.

11. The method of claim 1, comprising temporarily disabling one or more features of the analyte monitoring system after the occurrence of the predetermined signal characteristic is detected.

12. The method of claim 11, wherein the one or more features include a therapeutic recommendation based on the sensor data.

13. The method of claim 11, comprising:
receiving an indication of user input acknowledging the recommendation for a confirmation test; and
re-enabling the one or more features of the analyte monitoring system.

14. The method of claim 11, wherein the indication of the user input includes an indication of a completion of a confirmation test.

15. The method of claim 1, wherein the recommendation for the confirmation test is displayed on a display of the user interface.

16. The method of claim 1, wherein the sensor data is derived from interstitial fluid and the recommendation for a confirmation test is a recommendation for a blood-derived confirmation test.

17. The method of claim 1, wherein the analyte is glucose or a ketone body or both.

18. The method of claim 1, comprising removing the indication of the recommendation for the confirmation test after a predetermined condition occurs.

19. The method of claim 18, comprising:
detecting, with the processing circuitry, an occurrence of a subsequent predetermined signal characteristic in subsequent sensor data received; and
indicating, with the user interface, a subsequent recommendation for a confirmation test after the subsequent occurrence of the subsequent predetermined signal characteristic is detected.

20. The method of claim 18, wherein the predetermined condition is a completion of a confirmation test.

21. The method of claim 18, wherein the predetermined condition is a completion of a confirmation test resulting in a confirmation of accuracy.

22. The method of claim 18, wherein the predetermined condition is an absence of any predetermined signal characteristics in the sensor data.

23. The method of claim 18, wherein the predetermined condition is an elapse of a predetermined period of time.

24. The method of claim 23, wherein the predetermined period of time is between 1 and 3 hours after the detection of the predetermined signal characteristic.
